(19) United States
(12) Patent Application Publication
(10) Pub. No.: US 2013/0189780 A1
(43) Pub. Date: Jul. 25, 2013

(54) REPROGRAMMING COMPOSITIONS

(71) Applicants: Daniel Shoemaker, San Diego, CA (US); Peter Flynn, San Diego, CA (US)

(72) Inventors: Daniel Shoemaker, San Diego, CA (US); Peter Flynn, San Diego, CA (US)

(73) Assignee: FATE THERAPEUTICS, INC., San Diego, CA (US)

(21) Appl. No.: 13/519,558

(22) PCT Filed: Dec. 21, 2010
(86) PCT No.: PCT/US10/61615

§ 371 Date: Nov. 28, 2012

Related U.S. Application Data
(63) Continuation of application No. 61/291,709, filed on Dec. 31, 2009.

(51) Int. Cl. C12N 5/071

(52) U.S. Cl. CPC C12N 5/0696
USPC 435/375; 536/23.5; 530/300

(57) ABSTRACT

The present invention provides compositions and methods of using the compositions to alter the developmental potency of a cell. The present invention provides in vivo and ex vivo cell reprogramming or dedifferentiation methods suitable for autologous cell therapy and regenerative medicine.

### RELATED APPLICATIONS

This application claims the benefit under 35 U.S.C. §119(e) of U.S. Provisional Patent Application No. 61/291,709 filed Dec. 31, 2009, which is herein incorporated by reference in its entirety.

This patent application is related to U.S. Provisional Patent Application No. 61/241,647, filed Sep. 11, 2009, which is herein incorporated by reference in its entirety.

### BACKGROUND

1. Technical Field

The present invention relates generally to compositions and methods of using the same to increase the developmental potency of a cell. The present invention further provides compositions comprising one or more artificial pluripotency transcription factors and/or one or more small molecule reprogramming agents to increase cell potency.

2. Description of the Related Art

Groundbreaking work demonstrated that ectopic expression of four transcription factors, Oct-3/4, Klf-4, Sox-2, and c-Myc, could reprogram murine somatic cells to induced pluripotent stem cells (iPSCs) (Takahashi and Yamanaka, 2006; Wernig et al., 2007; Okita et al., 2007; Maherali et al., 2007), and human iPSCs were subsequently generated using similar genetic manipulation (Takahashi et al., 2007; Yu et al., 2007; Park et al., 2008; Lowrey et al., 2008). To address potential safety concerns, several groups attempted to reprogram somatic cells using different combinations of the four factors in combination with small molecules in an effort to eliminate the putative oncogenic effects of c-Myc, Sox-2, and Klf-4 and increase the efficiency of somatic cell reprogramming.

Wernig et al., 2008, described the reprogramming of MEFs using virally encoded Oct-3/4, Sox-2, and Klf-4. Nakagawa et al., 2008, disclose the reprogramming of both mouse and human fibroblasts using virally encoded Oct-3/4, Sox-2, and Klf-4. Huangfu et al., 2008, improved the somatic cell reprogramming of MEFs using virally encoded Oct-3/4, Sox-2, and Klf-4 in combination with the small molecule HDAC inhibitor valproic acid (VPA).

Huangfu et al., 2008, described somatic cell reprogramming of primary human fibroblasts with virally encoded Oct4 and Sox2 in combination with VPA. Silva et al., 2008, disclosed somatic cell reprogramming of mouse neural stem cells by using a two-step method. First the mouse neural stem cells were transduced with virally encoded Oct-3/4 and Klf-4 and subsequently, the transduced cells were cultured in a cell culture medium containing leukemia inhibitory factor (LIF), a MEK inhibitor, and a glycogen synthase kinase-3 (GSK3) inhibitor. Kim et al., 2008, disclosed reprogramming mouse neural stem cells with virally encoded Oct4 together with either Klf4 or c-Myc. WO2009/117439 demonstrated that MEFs could be reprogrammed using Oct 3/4 and Klf-4 alone; viral Oct 3/4 and Klf-4 and the small molecule BIX01294 (H3K9 methyltransferase inhibitor); and viral Oct 3/4 and Klf-4 in combination with the small molecules BIX01294 and BayK8644 (L-type Ca channel agonist). In addition, WO2009/117439 discloses the non-genetic reprogramming of MEFs using cell permeable versions of the reprogramming factors Oct 3/4, Sox2, Klf-4, optionally in combination with c-Myc.

Kim et al., 2009, described somatic cell reprogramming of mouse neural stem cells using only virally encoded Oct-3/4. Guo et al., 2009, disclosed reprogramming of EpiSCs using a two step process. The first step was to transfect mouse EpiSCs with a piggyBAC transposon carrying a Klf-4 transgene. The second step entailed culturing the transfected cell in a cell culture medium containing LIF, a MEK inhibitor and a GSK3 inhibitor.

Although strides have been made in reducing the number of genetic insults in reprogramming somatic cells; in most cases, the reprogrammed cells are not tested for complete pluripotency (e.g., germline transmission of rodent iPSCs) or the iPSCs fail to give rise to chimeric mice and are incompletely pluripotent. Or, in the case of human reprogramming experiments, the human iPSCs are able to form teratomas, but do not share any signaling properties with mouse embryonic stem cells, which are the gold standard animal model in the field.

Realization of the promise of iPSCs will require improved methods of directed differentiation for generating homogenous populations of lineage-specific cell types as well as elimination of the risks and drawbacks associated with the current iPSC protocols, including genetic manipulation, and the low-efficiency/slow kinetics of induction. Thus, there is still a need for non-genetic methods of reprogramming that increase the efficiency of iPSC generation and also increase the quality and developmental potency of iPSCs.

### BRIEF SUMMARY

In one embodiment, the present invention contemplates, in part, a method of increasing the potency of a cell, comprising contacting the cell with one or more polynucleotides, each comprising an artificial pluripotency transcription factor (APTF) wherein the APTF comprises polypeptide domains encoding a nuclear localization sequence (NLS), a DNA binding domain (DBD), and a transcriptional activation domain (TAD), wherein at least two of the polypeptide domains of the APTF are heterologous polypeptide domains, and wherein the contacting is performed under conditions and for a time sufficient, to induce at least one pluripotent stem cell characteristic in the cell, thereby increasing the potency of the cell.

In a particular embodiment, the APTF comprises a cell permeable peptide (CPP).

In another embodiment, the polynucleotide further comprises a vector. In a related embodiment, the polynucleotide further comprises one or more of a promoter, an enhancer, a 5′ untranslated region (UTR), a Kozak sequence, an intron, a polyadenylation sequence, a 3′UTR, and an epitope tag.

In certain embodiments, the APTF comprises a DBD selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

In another certain embodiment, the APTF comprises a DBD selected from the group consisting of: Oct-3/4, Nanog, Sox2, cMyc, Klf-4, Stat-3, Tcf-3, Stella, Rex-1, UTF-1, Dax-1, Nac-1, Sall4, TDGD-1, or Zfp-281.

In additional embodiments, the APTF comprises a DBD selected from the group consisting of: Oct-3/4, Nanog, Sox2, Klf-4, Stella, or Sall4.

In one preferred embodiment, the APTF comprises a DBD from Oct-3/4.

In particular embodiments, the APTF comprises a TAD selected from the group consisting of: VP16, VP64, SV40 Large T-antigen, E1A activation domain, relA, and EGFR-1.

In various embodiments the cell is contacted with one or more APTFs and with one or more small molecule reprogramming agents selected from the group consisting of: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGF receptor/ALK5 inhibitor, an HDAC inhibitor; an Erk inhibitor, a ROCK inhibitor, and an FGFR inhibitor.

In a particular embodiment, the potency of a multipotent cell is increased.

In another particular embodiment, the potency of a partially pluripotent cell is increased. In a certain embodiment, the partially pluripotent cell is an incompletely pluripotent iPSC or an EpiSC. In a related embodiment, the cell is contacted in a culture medium containing hLIF, an ALK5 inhibitor, a MEK inhibitor, and a GSK3 inhibitor.

In one embodiment, the present invention contemplates, in part, A method of increasing the potency of a cell, comprising contacting the cell with one or more artificial pluripotency transcription factor polypeptides, wherein each APTF polypeptide comprises polypeptide domains encoding a NLS, DBD, and a TAD, wherein at least two of the polypeptide domains of the APTF are heterologous polypeptide domains, and wherein the contacting is performed under conditions and for a time sufficient, to induce at least one pluripotent stem cell characteristic in the cell, thereby increasing the potency of the cell.

In a particular embodiment, the APTF comprises a cell permeable peptide (CPP).

In certain embodiments, the APTF comprises a DBD selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

In another certain embodiment, the APTF comprises a DBD selected from the group consisting of: Oct-3/4, Nanog, Sox2, cMyc, Klf-4, Stat-3, Tcf-3, Stella, Rex-1, UTF-1, Dax-1, Nac-1, Sall4, TDGD-1, or Zfp-281.

In additional embodiments, the APTF comprises a DBD selected from the group consisting of: Oct-3/4, Nanog, Sox2, Klf-4, Stella, or Sall4.

In one preferred embodiment, the APTF comprises a DBD from Oct-3/4.

In particular embodiments, the APTF comprises a TAD selected from the group consisting of: VP16, VP64, SV40 Large T-antigen, E1A activation domain, relA, and EGFR-1.

In various embodiments the cell is contacted with one or more APTFs and with one or more small molecule reprogramming agents selected from the group consisting of: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor.

In a particular embodiment, the potency of a multipotent cell is increased.

In another particular embodiment, the potency of a partially pluripotent cell is increased. In a certain embodiment, the partially pluripotent cell is an incompletely pluripotent iPSC or an EpiSC. In a related embodiment, the cell is contacted in a culture medium containing hLIF, an ALK5 inhibitor, a MEK inhibitor, and a GSK3 inhibitor.

In various embodiments, the present invention contemplates, in part, polynucleotides comprising one or more artificial pluripotency transcription factors (APTF), wherein each APTF comprises polypeptide domains encoding a NLS, DBD, and a TAD, wherein at least two of the polypeptide domains of the APTF are heterologous polypeptide domains

In a particular embodiment, an APTF polynucleotide comprises a cell permeable peptide (CPP).

In certain embodiments, an APTF polynucleotide comprises a DBD selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sault Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Left, Sox15, Sox6, Tcf-7, Tcf711, c-Myc, L-Myc, N-Myc, Hand1, Mad 1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

In another certain embodiment, an APTF polynucleotide comprises a DBD selected from the group consisting of: Oct-3/4, Nanog, Sox2, cMyc, Klf-4, Stat-3, Tcf-3, Stella, Rex-1, UTF-1, Dax-1, Nac-1, Sall4, TDGD-1, or Zfp-281.

In additional embodiments, an APTF polynucleotide comprises a DBD selected from the group consisting of: Oct-3/4, Nanog, Sox2, Klf-4, Stella, or Sall4.

In one preferred embodiment, an APTF polynucleotide comprises a DBD from Oct-3/4.

In particular embodiments, an APTF polynucleotide comprises a TAD selected from the group consisting of: VP16, VP64, SV40 Large T-antigen, E1A activation domain, relA, and EGFR-1.

In particular embodiments, an APTF polynucleotide comprises a protein-protein interaction domain (PPID), a ligand interacting domain (LID), one or more polypeptide linkers, and/or a polypeptide cleavage signal in any suitable combination.

In various embodiments, a polypeptide comprising one or more artificial pluripotency transcription factors (APTF), wherein each APTF comprises polypeptide domains encoding a NLS, DBD, and a TAD, wherein at least two of the polypeptide domains of the APTF are heterologous polypeptide domains

In a particular embodiment, an APTF polypeptide comprises a cell permeable peptide (CPP). In particular embodiments, an APTF polypeptide comprises a protein-protein interaction domain (PPID), a ligand interacting domain (LID), one or more polypeptide linkers, and/or a polypeptide cleavage signal in any suitable combination.

In certain embodiments, an APTF polypeptide comprises a DBD selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf₁, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Left, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

In another certain embodiment, an APTF polypeptide comprises a DBD selected from the group consisting of: Oct-3/4, Nanog, Sox2, cMyc, Klf-4, Stat-3, Tcf-3, Stella, Rex-1, UTF-1, Dax-1, Nac-1, Sall4, TDGD-1, or Zfp-281.

In additional embodiments, the APTF polynucleotide comprises a DBD selected from the group consisting of: Oct-3/4, Nanog, Sox2, Klf-4, Stella, or Sall4.

In one preferred embodiment, an APTF polypeptide comprises a DBD from Oct-3/4.

In particular embodiments, an APTF polypeptide comprises a TAD selected from the group consisting of: VP16, VP64, SV40 Large T-antigen, E1A activation domain, relA, and EGFR-1.

In one embodiment, the present invention provides a composition comprising a cell, one or more artificial pluripotency transcription factor polypeptides, one or more small molecule reprogramming agents, and a cell culture medium.

In another embodiment, the present invention provides a composition comprising a cell, one or more artificial pluripotency transcription factor polypeptides and one or more small molecule reprogramming agents.

In particular embodiments, compositions of the present invention comprise a multipotent cell. In certain embodiments, compositions of the present invention comprise a partially pluripotent cell. In one preferred embodiment, the partially pluripotent cell is an incompletely pluripotent iPSC or an EpiSC.

In an additional embodiment, the present invention provides a composition comprising one or more artificial pluripotency transcription factor polypeptides, one or more small molecule reprogramming agents, and a cell culture medium.

In a further embodiment, the present invention provides a composition comprising one or more artificial pluripotency transcription factor polypeptides and one or more small molecule reprogramming agents.

In various particular embodiments, the present invention provides a composition comprising an ATPF as described herein throughout and one or more small molecules as described herein throughout. In related embodiments, the composition comprises a cell.

### DETAILED DESCRIPTION

The present invention generally relates to improved compositions and methods for increasing cell potency and related therapeutic applications involving the same. More particularly, the present invention relates to compositions and methods for increasing cell potency by non-genetic reprogramming means. In various embodiments, increasing the developmental potency of a cell is achieved by contacting a cell with one or more engineered pluripotency transcription factors and/or a composition comprising one or more small molecule reprogramming agents.

The present invention contemplates, in part, to reprogram cells in vitro, in vivo or ex vivo, by modulation of specific cellular pathways, either directly or indirectly, using polynucleotide-, polypeptide- and/or small molecule-based approaches. As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “potency” refers to the sum of all developmental options accessible to the cell (i.e., the developmental potency). One having ordinary skill in the art would recognize that cell potency is a continuum, ranging from the most plastic cell, a totipotent stem cell, which has the most developmental potency to the least plastic cell, a terminally differentiated cell, which has the least developmental potency.

The continuum of cell potency includes, but is not limited to, totipotent cells, pluripotent cells, multipotent cells, oligopotent cells, unipotent cells, and terminally differentiated cells. In the strictest sense, stem cells are either totipotent or pluripotent; thus, being able to give rise to any mature cell type. However, multipotent, oligopotent or unipotent progenitor cells are sometimes referred to as lineage restricted stem cells (e.g., mesenchymal stem cells, adipose tissue derived stem cells, etc.) and/or progenitor cells.

As used herein, the term “totipotent” refers to the ability of a cell to form all cell lineages of an organism. For example, in mammals, only the zygote and the first cleavage stage blastomeres are totipotent.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell). The level of cell pluripotency can be determined by assessing pluripotency characteristics of the cells. Pluripotency characteristics include, but not limited to: i) pluripotent stem cell morphology; ii) expression of pluripotent stem cell markers including, but not limited to SSEA1, SSEA3/4; TRA1-60/81; TRA1-85, TRA2-54, GCTM-2, TG343, TG30, CD9, CD29, CD133/prominin, CD140a, CD56, CD73, CD105, CD31, CD34, OCT4, Nanog and/or Sox2; iii) ability of pluripotent stem cells to contribute to germline transmission in mouse chimeras; iv) ability of pluripotent stem cells to contribute to the embryo proper using tetraploid embryo complementation assays; v) teratoma formation of pluripotent stem cells; vi) formation of embryoid bodies: and vii) inactive X chromosome reactivation.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical intercell spacing.

As used herein, the term “multipotent” refers to the ability of an adult stem cell to form multiple cell types of one lineage. For example, hematopoietic stem cells are capable of forming all cells of the blood cell lineage, e.g., lymphoid and myeloid cells. As used herein, the term “oligopotent” refers to the ability of an adult stem cell to differentiate into only a few different cell types. For example, lymphoid or myeloid stem cells are capable of forming cells of either the lymphoid or myeloid lineages, respectively. As used herein, the term “unipotent” means the ability of a cell to form a single cell type. For example, spermatogonial stem cells are only capable of forming sperm cells.

### I. Overview

A number of cell signaling pathways may be important in increasing, establishing, and/or maintaining the potency of a cell. For example, developmental signal transduction pathways that can be important in regulating the pluripotency of a cell include, but are not limited to, a WNT pathway, a Hedgehog pathway, a Notch signaling pathway, receptor tyrosine kinase pathways, non-receptor tyrosine kinase pathways, PI3K/AKT pathways, Grb2/MEK pathways, MAPK/ERK pathways, TGF-β pathways, BMP pathways, GDF pathways, LIF pathways, Jak/Stat pathways, and Hox pathways.

In addition, particular transcription factors may be important for increasing, establishing, and/or maintaining the potency of a cell through transcription of overlapping sets of target genes. Exemplary transcription factors that are associated with increasing, establishing, or maintaining the potency of a cell include, but are not limited to Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf₁, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Left, Sox15, Sox6, Tcf-7, Tcf711, c-Myc, L-Myc, N-Myc, Hand 1, Mad 1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

Furthermore, several classes of small molecule reprogramming agents may be important to increasing, establishing, and/or maintaining the potency of a cell. Exemplary small molecule reprogramming agents include, but are not limited to: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor.

In one embodiment, the present invention contemplates, in part, a composition comprising one or more artificial pluripotency transcription factors that increases the potency of a cell and methods of using the same.

In a particular embodiment, the present invention contemplates, in part, a composition comprising one or more small molecule reprogramming agents that increases the potency of a cell and methods of using the same.

In another particular embodiment, the present invention contemplates, in part, a composition comprising one or more artificial pluripotency transcription factors in combination with one or more small molecule reprogramming agents that increases the potency of a cell and methods of using the same.

In a certain embodiment, the present invention contemplates, in part, to increase the potency of a partially pluripotent cell to a more primitive, more pluripotent cell, that is a cell with more developmental potency than the partially pluripotent cell. In a related embodiment, the incompletely pluripotent cell is an EpiSC and the more pluripotent cell has the potency of an embryonic stem cell.

### II. Cells of the Invention

The present invention contemplates, in part, to increase the potency of incompletely or partially pluripotent stem cells, multipotent cells, oligopotent cells, unipotent cells, and terminally differentiated cells. A suitable starting population of cells may be from any mammalian species. In particular embodiments, the starting population of cells is isolated from a mammal selected from the group consisting of: a rodent, a cat, a dog, a pig, a goat, a sheep, a horse, a cow, or a primate. In certain embodiments, the primate is a human.

A starting population of cells that is suitable for reprogramming or dedifferentiating according to the methods of the present invention, may be may be of any type of cell or a mixture of cell types. In one embodiment, the starting population of cells is selected from adult or neonatal stem/progenitor cells.

In particular embodiments, the starting population of stem/progenitor cells is selected from the group consisting of: mesodermal stem/progenitor cells, endodermal stem/progenitor cells, and ectodermal stem/progenitor cells.

In related embodiments, the starting population of stem/progenitor cells is a mesodermal stem/progenitor cell. Illustrative examples of mesodermal stem/progenitor cells include, but are not limited to: mesodermal stem/progenitor cells, endothelial stem/progenitor cells, bone marrow stem/progenitor cells, umbilical cord stem/progenitor cells, adipose tissue derived stem/progenitor cells, hematopoietic stem/progenitor cells (HSGs), mesenchymal stem/progenitor cells, muscle stem/progenitor cells, kidney stem/progenitor cells, osteoblast stem/progenitor cells, chondrocyte stem/progenitor cells, and the like.

In other related embodiments, the starting population of stem/progenitor cells is an ectodermal stem/progenitor cell. Illustrative examples of ectodermal stem/progenitor cells include, but are not limited to neural stem/progenitor cells, retinal stem/progentior cells, skin stem/progenitor cells, and the like.

In other related embodiments, the starting population of stem/progenitor cells is an endodermal stem/progenitor cell. Illustrative examples of endodermal stem/progenitor cells include, but are not limited to liver stem/progenitor cells, pancreatic stem/progenitor cells, epithelial stem/progenitor cells, and the like.

In certain embodiments, the starting population of cells may be a heterogeneous or homogeneous population of cells selected from the group consisting of: pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like.

### III. Artificial Pluripotency Transcription Factors

In preferred embodiments, compositions and methods that are used to increase the pluripotency of a cell include artificial pluripotency transcription factors (e.g., fusion polypeptides). In one embodiment, a cell is contacted with at least one artificial pluripotency transcription factor under conditions and for a time sufficient to increase the potency of the cell. The increase in potency is objectively measured using the criteria set forth above for assaying the pluripotency characteristics of a cell.

In particular embodiments, incompletely pluripotent human stem cells are contacted with one or more artificial pluripotency transcription factors thereby increasing the pluripotency of the cell to a more primitive pluripotent state. In a certain embodiment, the incompletely pluripotent cells are hiPSCs or hEpiSCs.

As used herein, the term “Artificial Pluripotency Transcription Factor” or “APTF” refers to an artificially designed transcription factor comprising at least two, three, four, five, six, seven, eight, nine, or ten fused heterologous polypeptide domains. In particular embodiments, contacting a cell with a composition comprising one or more APTFs increases, establishes, and/or maintains the developmental potency of the cell.

As used herein, the term “fused” refers to a biomolecule (e.g., polynucleotide or polypeptide) in which two or more subunit biomolecules are linked, preferably covalently. The subunit molecules can be the same chemical type of molecule, or can be different chemical types of molecules. Examples include, without limitation, fusion polypeptides (e.g., a DNA-binding domain fused to a transcriptional activation domain) and fusion polynucleotides (e.g., a polynucleotide encoding a fusion polypeptide described herein).

As used herein, “heterologous polypeptide” refers to two or more domains of a fusion polypeptide. A heterologous polypeptide indicates that two or more domains or segments of the polypeptide are not found in the same relationship to each other in nature, e.g., a fusion polypeptide comprising a DNA binding domain from a first polypeptide fused to a transcriptional activation domain from a second polypeptide. Similarly, the term “heterologous polynucleotide” refers to a nucleic acid comprising two or more subsequences that are not found in the same relationship to each other in nature, e.g., a polynucleotide encoding a heterologous polypeptide.

In some embodiments, an artificial pluripotency transcription factor includes two or more heterologous polypeptides and also includes two or more non-heterologous polypeptides.

Moreover, in particular embodiments, polynucleotides used to express recombinant polypeptides further include one or more additional regulatory polynucleotide sequences, e.g., vector polynucleotide sequences, promoters, enhancers, introns, 5′ and 3′ UTRs, and polyadenylation sequences.

Exemplary polypeptide domains or segments include, but are not limited to: cell permeable peptides (CPP), nuclear localization sequences (NLS), DNA binding domains (DBD), transcriptional activation domains (TAD), protein-protein interaction domains (PPID), ligand interacting domains (LIDs), other regulatory or enzymatic domains, and epitope tags. Additional polypeptide domains or segments include polypeptide linkers and polypeptide cleavage signals.

In particular embodiments, it is preferred that artificial pluripotency transcription factors are produced by fusion of a DBD and a TAD. In certain embodiments, it is preferred that artificial pluripotency transcription factors are produced by fusion a DBD and a TAD, and in addition, one or more NLSs, CPPs, PPIDs, LIDs, other regulatory or enzymatic domains, epitope tags, polypeptide linkers, and polypeptide cleavage signals

As used herein, the term “obtained from” when used in the context of obtaining a particular domain (e.g., DBD, TAD, NLS, CPP, PPID, LID) from a polypeptide or protein refers to identifying the sequence of the particular domain and incorporating it into an artificial pluripotency transcription factor using standard molecular biology techniques. Any suitable method known in the art can be used to design and construct nucleic acids encoding APTFs, e.g., phage display, random mutagenesis, combinatorial libraries, computer/rational design, affinity selection, PCR, cloning from cDNA or genomic libraries, synthetic construction, and the like.

A. Cell Permeable Peptides (CPP)

In various embodiments, an artificial pluripotency transcription factor comprises one or more CPPs. An important factor in the administration of polypeptide compounds is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins, lipids and other compounds, which have the ability to translocate polypeptides across a cell membrane, have been described.

Examples of peptide sequences which can facilitate protein uptake into cells include, but are not limited to: HIV TAT polypeptides; a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). In addition, Several bacterial toxins, including Clostridium perfringens iota toxin, diphtheria toxin (DT), Pseudomonas exotoxin A (PE), Bordetella pertussis toxin (PT), Bacillus anthracis toxin, and Bordetella pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193.

Other exemplary CPP amino acid sequences include, but are not limited to: RKKRRQRRR, KKRRQRRR, and RKKRRQRR (derived from HIV TAT protein); RRRRRRRRR; KKKKKKKKK; RQIKIWFQNRRMKWKK (from Drosophila Antp protein); RQIKIWFQNRRMKSKK (from Drosophila Ftz protein); RQIKIWFQNKRAKIKK (from Drosophila Engrailed protein); RQIKIWFQNRRMKWKK (from human Hox-A5 protein); and RVIRVWFQNKRCKDKK (from human Isl-1 protein).

Such subsequences can be used to facilitate polypeptide translocation, including the APTF polypeptides disclosed herein, across a cell membrane. This is accomplished, for example, by derivatizing the fusion polypeptide (e.g., APTF) with one or more CPP sequences or by forming an additional fusion of the CPP sequence with the fusion polypeptide. Optionally, a linker can be used to link the fusion polypeptide and the one or more CPP polypeptides. Any suitable linker can be used, e.g., a peptide linker, as described elsewhere herein.

Other suitable chemical moieties that provide enhanced cellular uptake can also be linked, either covalently or non-covalently, to a polypeptide as described herein.

B. Nuclear Localization Sequences (NLS)

In various embodiments, an artificial pluripotency transcription factor comprises one or more NLSs. A nuclear localization sequence is cellular targeting sequence which provides for the protein to be translocated to the nucleus. Typically a nuclear localization sequence has a plurality of basic amino acids, referred to as a bipartite basic repeat (reviewed in Garcia-Bustos et al., Biochimica et Biophysica Acta (1991) 1071, 83101). The NLS can be located in any part of an APTF polypeptide internal or proximal to the N- or C-terminus and results in the polypeptide being localized inside the nucleus. In particular embodiments, one or more NLS polypeptide sequences are introduced into a single APTF polypeptide.

Exemplary NLS sequences include, but are not limited to, PKKKRKV (from SV40 Large T-antigen), K(K/R)X(K/R) (from c-Myc), and residues 316-325, 369-375, and 379-384 of p53 (Shaulsky et al., 1990). The NLS of nucleoplasmin, KR[PAATKKAGQA]KKKK, is the prototype of ubiquitous bipartite signal: two clusters of basic amino acids, separated by a spacer of about 10 amino acids.

C. DNA Binding Domains (DBD)

As used herein, the term “pluripotency factor” refers to a pluripotency gene or a pluripotency polypeptide. Pluripotency genes and polypeptides are those that are associated with increasing, establishing, or maintaining pluripotency. The expression of a pluripotency gene is typically restricted to pluripotent stem cells, and is crucial for the functional identity of pluripotent stem cells.

In various embodiments, an artificial pluripotency transcription factor comprises one or more DBDs. Exemplary types of DBDs that can be used in artificial pluripotency transcription factors include, but are not limited to homeodomains, leucine lipper domains, HMG-box domains, forkhead/winged helix domains, basic Helix-Loop-Helix domains, Helix-Turn-Helix domains, T-box domains, and zinc finger domains.

In preferred embodiments, the DBDs are obtained from pluripotency polypeptides that are transcription factors (i.e., pluripotency transcription factors). A number of pluripotency transcription factors have been shown to be important for increasing, establishing, and/or maintaining the pluripotency of a cell. Thus, by including one or more DBDs from pluripotency transcription factors, the APTFs of the invention will be recuited to and activate transcription from genes important for increasing, establishing, and/or maintaining the pluripotency of a cell.

Exemplary pluripotency transcription factors from which DBDs can be obtained include, but are not limited to: homeodomain containing polypeptides, such as, for example, Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, and the like; zinc finger domain containing polypeptides, such as, for example, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, and the like; HMG-box domain containing polypeptides, such as, for example, Sox2, Left, Sox15, Sox6, Tcf-7, Tcf711, and the like; bHLH domain containing proteins, such as, for example, c-Myc, L-Myc, N-Myc, Hand1, Mad1, Mad3, Mad4, Mxi1, Myf5, Neurog2, Ngn3, Olig2, Tcf3, Tcf4, and the like; forkhead/winged helix domain polypeptides, such as, for example, Foxc1 and Foxd3 and the like; HTH domain containing polypeptides, such as BAF155 and the like; leucine zipper domain containing polypeptides such as, for example, C/EBPβ and mafa and the like; T-box domain containing polypeptides such as, for example, Eomes and Tbx-3; RFX domain containing polypeptides such as Rfx4 and the like; STAT domain containing polypeptides such as Stat3 and the like; and DBDs from other pluripotency transcriptions factors such as, for example, Stella and UTF-1

In further embodiments, the DBD can be an artificially engineered, homeodomain, leucine lipper, HMG-box, forkhead/winged helix, bHLH, HTH, T-box, or zinc finger domain that are design to recognize a particular sequence of polynucleotides in a target gene promoter. Such artificially designed sequence specific DBDs are well within the purview of the skilled artisan. See, e.g., WO 00/41566 and WO 00/42219. Additional exemplary disclosure regarding engineered zinc finger domains is described in U.S. Provisional Patent Application No. 61/241,647, the disclosure of which is herein incorporated by reference

In preferred embodiments, the DBD is obtained from Oct-3/4, Nanog, Sox2, cMyc, Klf-4, Stat-3, Tcf-3, Stella, Rex-1, UTF-1, Dax-1, Nac-1, Sall4, TDGD-1, or Zfp-281.

In another preferred embodiment, the DBD is obtained from Oct-3/4, Nanog, Sox2, Klf-4, Stella, or Sall4.

In yet another preferred embodiment, the DBD is obtained from Oct-3/4.

In other embodiments, an APTF comprises one, two, three, four, or more DBDs, optionally separated by linker polypeptides as described elsewhere herein. Spatial separation of the multiple DBDs allows for greater specificity and less interference from neighboring DBDs.

In one embodiment, an APTF comprises any two or three DBDs obtained from Oct-3/4, Nanog, Sox2, and Klf-4.

D. Transcriptional Activation Domains (TAD)

In various embodiments, an artificial pluripotency transcription factor comprises one or more TADs. TADs are important for increasing or potentiating transcription at any given genetic locus. TADs can comprise naturally-occurring or non-naturally-occurring polypeptide sequences, so long as they are capable of activating or potentiating transcription of a target gene. A variety of polypeptides and polypeptide sequences which can activate or potentiate transcription in eukaryotic cells are known and in many cases have been shown to retain their activation function when expressed as a component of a fusion protein.

In one embodiment, an APTF that increases, establishes, and/or maintains the pluripotency of a cell comprises the HSV VP16 activation domain (see, e.g., Hagmann et al., J. Virol. 71:5952-5962 (1997)); the VP64 activation domain (Seipel et al., EMBO J. 11:4961-4968 (1996)); a nuclear hormone receptor activation domain (see, e.g., Torchia et al., Curr. Opin. Cell. Biol. 10:373-383 (1998)); the SV40 Large T-antigen activation domain (Johnston et al. J. Virol. 1996 February; 70(2): 1191-1202); the E1A activation domain (Lee et al., Cell, Volume 67, Issue 2, 365-376, 18 Oct. 1981); the activation domain from the p65 subunit of nuclear factor kappa B (Bitko & Barik, J. Virol. 72:5610-5618 (1998) and Doyle & Hunt, Neuroreport 8:2937-2942 (1997)); or the EGR-1 activation domain (early growth response gene product-1; Yan et al., PNAS 95:8298-8303 (1998); and Liu et al., Cancer Gene Ther. 5:3-28 (1998)).

Additional exemplary activation domains include, but are not limited to those identified in, p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, API, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Further exemplary transcriptional activation domains include acidic transcription activation domains (noted previously), proline-rich transcription activation domains, serine/threonine-rich transcription activation domains, and glutamine-rich transcription activation domains. Non-limiting examples of proline-rich activation domains include amino acid residues 399-499 of CTF/NF1 and amino acid residues 31-76 of AP2. Non-limiting examples of serine/threonine-rich transcription activation domains include amino acid residues 1-427 of ITF1 and amino acid residues 2-451 of ITF2. Non-limiting examples of glutamine-rich activation domains include amino acid residues 175-269 of Oct-1 and amino acid residues 132-243 of Sp1.

Still other illustrative activation domains and motifs of human origin include the activation domain of human CTF, the 18 amino acid (NFLQLPQQTQGALLTSQP) glutamine rich region of Oct-2, the N-terminal 72 amino acids of p53, the SYGQQS repeat in Ewing sarcoma gene and an 11 amino acid (535-545) acidic rich region of rel A protein.

One of skill in the art would appreciate that the strength of a given transcriptional activation domain can be increased or decreased through routine mutagenesis of selected residues within the activation domain. The effects of such mutations can be assayed in vitro using a transcriptional reporter assay, such as a choline acteyltransferase (CAT) assay or luciferase assay.

In one embodiment, an APTF comprises one or more DBDs selected from the group consisting of: Oct-3/4, Nanog, Sox2, Klf-4, Stella, and Sall4; and comprises one or more transcriptional activation domains selected from the group consisting of: VP16, VP64, SV40 Large T-antigen, E1A activation domain, relA, and EGFR-1.

In a particular embodiment, the APTF comprises one or more CPP polypeptides and one or more NLSs.

E. Protein-Protein Interaction Domains (PPID)

In particular embodiments, APTFs comprise a protein-protein interaction domain which enables the binding of an APTF to another protein molecule. For example, Klf-4 is known to interact with the Oct-3/4 and Sox2 complex (Wei et al., 2009); thus, an APTF comprising the protein interaction domain in Klf-4 that interacts with the Oct-3/4 and Sox2 would be expected to recruit these proteins in the cell. Similarly, Sox2 interacts with Oct-3/4 (Remenyi et al., Genes Dev. 2003 Aug. 15; 17(16):2048-59) and Nanog also interacts with Oct-3/4 (Wang et al., 2006). Thus, any protein interaction domains mediating the foregoing protein interactions would be expected to recruit the interacting protein partner in the cell.

In other embodiments, an APTF comprises a protein-protein interaction domain that allows the APTF to bind and recruit transcriptional co-activators, such as C/EBP and p300 and histone acetyltransferases and the like, to the site of transcription.

F Ligand Interacting Domains (LIDs)

In particular embodiments, an APTF that increases, establishes, and/or maintains the pluripotency of a cell comprises one or more ligand interacting domains. Generally, when a bipartite strategy is employed, a first APTF fragment comprising at least a DBD a first ligand interaction domain binds, in a ligand dependent matter, to a second APTF fragment comprising the second ligand interaction domain and a transcriptional activation domain. See Spencer, D. M., et al. 1993. Science. 262:1019-1024, and PCT/US94/01617. For example, in a partitite strategy, the first APTF fragment comprises an Oct-3/4 DBD fused to an FKBP12 ligand binding domain and the second APTF fragment comprises an FKBP12 ligand binding domain and a VP16 transcriptional activation domain. When the divalent ligand, FK1012 is added in the presence of the two APTF fragments, they dimerize via an FKBP12-FK1012-FKBP12 interaction.

In other embodiments, ligand interacting domains can be used to control the temporal activity of an APTF that increases, establishes, and/or maintains the pluripotancy of a cell.

In particular illustrative embodiments, a steroid hormone inducible hormone-binding domain (HBD) is fused to a heterologous APTF. Without wishing to be bound to any particular theory, in the absence of hormone, the HBD-APTF fusion protein is held in an inactive state, presumably due to complex formation with hsp 90 (Scherrer et al., 1993). Addition of hormone causes a conformational change that dissociates hsp90, resulting in the rapid activation of the APTF fusion protein (Tsai and O'Malley, 1994). Maximal temporal regulation of an HBD transcription factor fusion polypeptide is achieved when the HBD is fusion relatively close to the functional domain to be regulated (Mattioni et al., 1994; Picard D, Salser S J, and Yamamoto K R. Cell. 1988 Sep. 23; 54(7):1073-80; Godowski P J, Picard D, and Yamamoto K R. Science. 1988 Aug. 12; 241(4867):812-6).

Exemplary HBD-ligand pairs include, but are not limited to: the ER hormone binding domain—tamoxifen, the PR hormone binding domain—RU486, the GR hormone binding domain—dexamethasone, and the ecdysone receptor hormone binding domain—myristerone. In certain embodiments, the HBD is mutated to increase hormone ligand specificity.

G. Epitope Tags

In certain embodiments, an APTF comprises an epitope tag. The tag polypeptide has enough residues to provide an epitope against which an antibody can be made, yet is short enough such that it does not interfere with activity of the polypeptide to which it is fused. The tag polypeptide is also preferably fairly unique so that the antibody does not substantially cross-react with other epitopes. Suitable tag polypeptides generally have at least six amino acid residues and usually between about 8 and 50 amino acid residues (preferably, between about 10 and 20 amino acid residues). In various other embodiments, the APTF polypeptide is conjugated to an epitope tag selected from the group consisting of: maltose binding protein (“MBP”), glutathione S transferase (GST), HIS6, MYC, FLAG, V5, VSV-G, and HA.

Various tag polypeptides and their respective antibodies are well known in the art. Examples include poly-histidine (HIS6; poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985); and the Herpes Simplex virus glycoprotein D (gD) tag and its antibody (Paborsky et al., Protein Engineering, 3(6):547-553 (1990)). Another example is the FLAG-peptide (Hopp et al., BioTechnology, 6:1204-1210 (1988)), which is recognized by an anti-FLAG M2 monoclonal antibody (Sigma, St. Louis, Mo.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Examples of other tag polypeptides include the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

In one embodiment, an APTF polypeptide comprises an epitope tag for purification. In particular embodiments, the epitope tag may be rendered cleavable, either self-cleavable or chemically cleavable.

H. Linkers

Artificial pluripotency transcription factors can comprise one or more linker domains between each of the polypeptide domains described herein, e.g., between any combination of CPP, NLS, DBD, TAD, PPID, LID, other regulatory or enzymatic domains, and epitope tags.

A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: DGGGS; TGEKP (see, e.g., Liu et al., PNAS 5525-5530 (1997)); GGRR (Pomerantz et al. 1995, supra); (GGGGS)ₙ (Kim et al., PNAS 93, 1156-1160 (1996.); EGKSSGSGSESKVD (Chaudhary et al., 1990, Proc. Natl. Acad. Sci. U.S.A. 87:1066-1070); KESGSVSSEQLAQFRSLD (Bird et al., 1988, Science 242:423-426), GGRRGGGS; LRQRDGERP; LRQKDGGGSERP; LRQKd(GGGS)₂ ERP. Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

I. Polypeptide Cleavage Signals

Artificial pluripotency transcription factors can comprise a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Targeting of proteins derived from self-processing polyproteins containing multiple signal sequences. Traffic, August; 5(8); 616-26).

Suitable protease cleavages sites and self-cleaving peptides are known to the skilled person (see, e.g., in Ryan et al. (1997) J. Gener. Virol. 78, 699-722; Scymczak et al. (2004) Nature Biotech. 5, 589-594). Exemplary protease cleavage sites include, but are not limited to the cleavage sites of potyvirus Nla proteases (e.g., tobacco etch virus protease), potyvirus HC proteases, potyvirus P1 (P35) proteases, byovirus Nla proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S), for example, ENLYFQG and ENLYFQS, wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In particular embodiments, polynucleotide constructs of the present invention can encode for more than one artificial pluripotency transcription factors in a single transcript. For example, in other particular embodiments a polynucleotide encodes for two, three, four, five or more APTFs in a single transcript. In certain embodiments, each of the polynucleotides encoding a separate APTF is separated by a polynucleotide encoding a self-cleaving polypeptide. Without wishing to be bound to any particular theory, once the multi-cistronic APTF is translated, the polypeptide comprising the two or more APTFs separated by self-cleaving polypeptides, will be cleaved into the separate APTFs.

### IV. Polypeptides

In various embodiments, compositions that are used to increase, establish and/or maintain the pluripotency of a cell include polypeptides as described herein (e.g., an artificial pluripotency transcription factor) as well as polynucleotides encoding the same.

As used herein, the terms “polypeptide,” “peptide,” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. Polypeptides can be prepared using any of a variety of well known recombinant and/or synthetic techniques, illustrative examples of which are further discussed below.

Polypeptides include polypeptide variants. Polypeptide variants may differ from a naturally occurring polypeptide in one or more substitutions, deletions, additions and/or insertions. Such variants may be naturally occurring or may be synthetically generated, for example, by modifying one or more of the above polypeptide sequences used in the methods of the invention and evaluating their effects using any of a number of techniques well known in the art. Preferably, polypeptides of the invention include polypeptides having at least about 65%, 70%, 75%, 85%, 90%, 95%, 98%, or 99% amino acid identity thereto.

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with an ability to modulate, induce and/or maintain pluripotency as described herein. When it is desired to alter the amino acid sequence of a polypeptide to create an equivalent, or even an improved, variant polypeptide of the invention, one skilled in the art, for example, can change one or more of the codons of the encoding DNA sequence, e.g., according to Table 1.

TABLE 1
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic | D | Asp | GAC GAU |
| acid | | | |
| Glutamic | E | Glu | GAA GAG |
| acid | | | |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG |
| | | | CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG |
| | | | CGU |
| Serine | S | Ser | AGC AGU UCA UCC UCG |
| | | | UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Exemplary conservative substitutions are described in U.S. Provisional Patent Application No. 61/241,647, the disclosure of which is herein incorporated by reference.

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. Examples of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively.

A. Fusion Polypeptides

Polypeptides of the present invention include fusion polypeptides (e.g., artificial pluripotency transcription factors). In preferred embodiments, fusion polypeptides and polynucleotides encoding fusion polypeptides are provided. Fusion polypeptides and fusion proteins refer to a polypeptide having at least two, three, four, five, six, seven, eight, nine, or ten heterologous polypeptide segments.

The polypeptide domains or segments (e.g., CPP, NLS, DBD, TAD, PPID, LIDs, other regulatory or enzymatic domains, epitope tags, polypeptide linkers, and polypeptide cleavage signals) forming the fusion polypeptide are typically typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, and interspecies homologs, so long as the desired transcriptional activity of the fusion polypeptide is preserved.

Amino acids in polypeptides of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as binding to a natural or synthetic binding partner (e.g., polynucleotide transcription factor binding site; EMSA assays). Furthermore, transcriptional activity of fusion polypeptides, mutants, and variants thereof can be assayed in vitro using CAT or luciferase reporter assays as generally described in the art. Sites that are critical for protein-DNA binding can also be determined by structural analysis such as crystallization, nuclear magnetic resonance or photoaffinity labeling (Smith et al., J. Mol. Biol. 224:899-904, 1992 and de Vos et al. Science 255:306-312, 1992). Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support.

The fusion partner may be designed and included for essentially any desired purpose provided they do not adversely affect the desired activity of the polypeptide. For example, in one embodiment, a fusion protein may be designed to emulate the transcriptional activity of multiple pluripotency transcription factors. In another embodiment, two or more distinct artificial plurpotency factors separated by polypeptide cleavage signals may be translated from the same mRNA and, when translated, the multimeric APTF undergoes a self-cleavage reaction to generate the individual monomeric artificial pluripotency factors.

In another embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments. Still further fusion partners include affinity tags, which facilitate purification of the protein. Fusion polypeptides of the present invention also include, but are not limited to artificially designed transcription factors, as described elsewhere herein.

Fusion polypeptides may be produced by chemical synthetic methods or by chemical linkage between the two moieties or may generally be prepared using other standard techniques. In particular embodiments, it is preferred that fusion polypeptides are produced by fusion of a DBD and a TAD. In certain embodiments, it is preferred that fusion polypeptides are produced by fusion a DBD and a TAD, and in addition, one or more NLSs, CPPs, PPIDs, LIDs, other regulatory or enzymatic domains, epitope tags, polypeptide linkers, and polypeptide cleavage signals

The ligated DNA sequences are operably linked to suitable transcriptional or translational regulatory elements. The regulatory elements responsible for expression of DNA are located 5′ to the DNA sequence encoding the first polypeptide or within a natural or non-natural intron. Similarly, stop codons required to end translation and transcription termination signals are present 3′ to the DNA sequence encoding the second polypeptide or within an intron.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

### V Polynucleotides

In one embodiment, isolated polynucleotides that encode polypeptides or fusion polypeptides of the invention (e.g., an artificial pluripotency transcription factor) are provided. A cell contacted with a polynucleotide encoding an artificial pluripotency transcription factor has an increased level of developmental potency compared to a cell that has not been contacted a polynucleotide encoding an artificial pluripotency transcription factor. Thus, a polynucleotide encoding an artificial pluripotency transcription factors increases, establishes, or maintains the pluripotency of a cell.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, BACs, piggyback transposons, and the like. Nucleotides of the present invention include inosine, adenine, guanine, cytosine, thymine, uracil, analogs or derivative thereof and the like.

Preferably, polynucleotides of the invention include polynucleotides having at least about 65%, 70%, 75%, 85%, 90%, 95%, 98%, or 99% nucleotide identity thereto.

As will be understood by those skilled in the art, the polynucleotide sequences include genomic sequences, extra-genomic and plasmid-encoded sequences and smaller engineered gene segments that express, or may be adapted to express, proteins, polypeptides, peptides, and the like. Such segments may be naturally isolated, recombinant, or modified synthetically by the hand of man.

As will be recognized by the skilled artisan, polynucleotides may be single-stranded (coding or antisense) or double-stranded, and may be DNA (genomic, cDNA or synthetic) or RNA molecules. Polynucleotides may comprise a native sequence (i.e., an endogenous sequence that encodes a polypeptide or fusion polypeptide of the invention or a portion thereof) or may comprise a variant, or a biological functional equivalent of such a sequence. Polynucleotide variants may contain one or more substitutions, additions, deletions and/or insertions, as further described below, preferably such that ability of the encoded polypeptide to increase, establish, and/or maintain the pluripotency of a cell is not substantially diminished relative to the unmodified polypeptide.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

Polynucleotides can be constructed by various amplification methods. One of the best known amplification methods is the polymerase chain reaction (PCR™) which is described in detail in U.S. Pat. Nos. 4,683,195, 4,683,202 and 4,800,159, each of which is incorporated herein by reference in its entirety.

Other illustrative amplification methods include the ligase chain reaction (referred to as LCR), Qbeta Replicase, Strand Displacement Amplification (SDA), Repair Chain Reaction (RCR), transcription-based amplification systems (TAS), and nucleic acid sequence based amplification (NASBA).

In certain instances, it is possible to obtain a full length cDNA or a relevant segment by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

Polynucleotides can be prepared, manipulated and/or expressed using any of a variety of well established techniques known and available in the art. For example, polynucleotide sequences which encode polypeptides described herein, can be used in recombinant DNA molecules to direct expression of the polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

In order to express a desired polypeptide, a nucleotide sequence encoding the polypeptide, can be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression prokaryotic and eukaryotic vector/host systems are known and may be utilized to contain and express polynucleotide sequences. These include, but are not limited to, bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including constitutive promoters (e.g., CMV, Ubiquitin C, and EF1a) and inducible promoters (e.g., tetracycline regulated), may be used.

A polypeptide of the invention may be produced recombinantly not only directly, but also as a fusion polypeptide comprising a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native polypeptide signal sequence, the signal sequence is substituted by a prokaryotic signal sequence selected, for example, from the group of the alkaline phosphatase, penicillinase, Ipp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2p plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli. Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt− cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). Trp1 and/or Leu2 deficient yeast strains provide a selection marker for a mutant strain of yeast lacking the ability to grow in tryptophan (e.g., ATCC No. 44076 or PEP4-1) or leucine (e.g., ATCC 20,622 or 38,626).

Expression and cloning vectors generally contain a promoter that is recognized by the host organism and is operably linked to nucleic acid encoding a polypeptide. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno sequence operably linked to the DNA encoding a polypeptide. The Shine Dalgarno sequence, AGGAGG, is usually located 4-7 nucleotides 5′ of the initiator AUG of many mRNAs. Exemplary bacterial cloning and expression vectors include, but are not limited to pBluescript® (Strategene), pET® (Pharmacia), pUC-19 (Promega), pBR22 (NEB), pGEX® (Promega), pIN vectors (Van Heeke & Schuster, J. Biol. Chem. 264:5503 5509 (1989)).

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. Exemplary eukaryotic promoters include, but are not limited to: Polypeptides transcribed from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), Ubiquitin C, and EF1a or from other heterologous mammalian promoters, e.g., the actin promoter or an immunoglobulin promoter, from heat-shock promoters, and the like, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is preferably located at a site 5′ from the promoter.

Most eukaryotic mRNAs contain a short recognition sequence that greatly facilitate the initial binding of mRNA to the small subunit of the ribosome. The consensus sequence for initiation of translation in vertebrates (also called Kozak sequence is (GCC)RCCATGG, where R is a purine (A or G) (Kozak, M. Cell. 1986, 44(2):283-92, and Kozak, M. Nucleic Acids Res. 1987, 15(20):8125-48).

At the 3′ end of most eukaryotic genes is an AATAAA sequence that may be the signal for addition of the poly A tail to the 3′ end of the coding sequence. All of these sequences are suitably inserted into eukaryotic expression vectors.

Host cell strains may be chosen for their ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as 3T3, M2-10B4 cells, C3H10T1/2 cells, CHO, HeLa, MDCK, HEK293, and W138, which have specific cellular machinery and characteristic mechanisms for such post-translational activities, may be chosen to ensure the correct modification and processing of the foreign protein.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Exemplary accession numbers for polynucleotide and polypeptide sequences of the genes/proteins associated with pluripotency factors include, bt are not limited to: Ac133 (e.g., NM_{—}001145852, NM_{—}001145851, N_{—}001145850, NM_{—}001145849, NM_{—}001145848, NM_{—}001145847, NM_{—}006017, NP_{—}001139324, NP_{—}001139323, NP_{—}001139322, NP_{—}001139321, NP_{—}001139320, NP_{—}001139319, NP_{—}006008); Alp (e.g., NM_{—}207303 and NP_{—}997186); Atbf1 (e.g., NM_{—}006885 and NP_{—}008816); Axin2 (e.g., NM_{—}004655 and NP_{—}004646); BAF155 (e.g., NM_{—}003074 and NP_{—}003065); bFgf (e.g., NM_{—}002006 and NP_{—}001997); Bmi1 (e.g., NM_{—}005180 and NP_{—}005171); Boc (e.g., NM_{—}033254, NP_{—}150279); C/EBPβ(e.g., NM_{—}005194 and NP_{—}005185); CD9 (e.g., NM_{—}001769 and NP_{—}001760); Cdon (e.g., NM_{—}016952 and NP_{—}058648); Cdx-2 (e.g., NM_{—}001265 and NP_{—}001256); c-Kit (e.g., NM_{—}000222, NM_{—}001093772, NP_{—}001087241, and NP_{—}000213); c-Myc (e.g., NM_{—}002467 and NP_{—}002458); Coup-Tf1 (e.g., NM_{—}005654 and NP_{—}005645); Csl (e.g., NM_{—}022579, NM_{—}022580, NM_{—}022581, NM_{—}001318, NP_{—}001309, NP_{—}072103, NP_{—}072102, and NP_{—}072101); Ctbp (e.g., NM_{—}203292, NM_{—}203291, NM_{—}002894, NP_{—}976037, NP_{—}976036, and NP_{—}002885); Dax1 (e.g., NM_{—}000475 and NP_{—}000466); Dnmt3A (e.g., NM_{—}175630, NM_{—}175629, NM_{—}153759, NM_{—}022552, NP_{—}715640, NP_{—}783329, NP_{—}783328, and NP_{—}072046); Dnmt3B (e.g., NM_{—}175850, NM_{—}175849, NM_{—}175848, NM_{—}006892, NP_{—}787046, NP_{—}787045, NP_{—}787044, and NP_{—}008823); Dnmt3L (e.g., NM_{—}175867, NM_{—}013369, NP_{—}787063, and NP_{—}037501); Dppa2 (e.g., NM_{—}138815 and NP_{—}620170); Dppa4 (e.g., NM_{—}018189 and NP_{—}060659); Dppa5 (e.g., NM_{—}001025290 and NP_{—}001020461); Ecat1 (e.g., NM_{—}001017361 and N_{—}001017361); Ecat8 (e.g., NM_{—}001110822 and NP_{—}001104292); Eomes (e.g., NM_{—}005442 and NP_{—}005433); Eras (e.g., NM_{—}181532 and NP_{—}853510); Esg1 (e.g., NM_{—}005077 and NP_{—}005068); Esrrb (e.g., NM_{—}004452 and NP_{—}004443); Fbx15 (e.g., NM_{—}001142958, NM_{—}152676, NP_{—}001136430, and NP_{—}689889); Fgf4 (e.g., NM_{—}002007 and NP_{—}001998); Flt3 (e.g., NM_{—}004119 and NP_{—}004110); Foxc1 ((e.g., NM_{—}001453 and NP_{—}001444); Foxd3 (e.g., NM_{—}012183 and NP_{—}036315); Fzd9 (e.g., NM_{—}003508 and NP_{—}003499); Gbx2 (e.g., NM_{—}001485 and NP_{—}001476); Gcnf (e.g., NM_{—}033334, NM_{—}001489, NP_{—}001480, and NP_{—}201591); Gdf10 (e.g., NM_{—}004962 and NP_{—}004953); Gdf3 (e.g., NM_{—}020634 and NP_{—}065685); Gdf5 (e.g., NM_{—}000557 and NP_{—}000548); Grb2 (e.g., NM_{—}203506, NM_{—}002086, NP_{—}002077, and NP_{—}987102); Groucho (e.g., NM_{—}005077, NP_{—}005068, NM_{—}007005, NP_{—}008936, NM_{—}001105192, NM_{—}020908, NM_{—}005078, NP_{—}001098662, NP_{—}065959, NP_{—}005069, NM_{—}001144762, NM_{—}001144761, NM_{—}003260, NP_{—}001138234, NP_{—}001138233, and NP_{—}003251); Gsh1 (e.g., NM_{—}145657 and NP_{—}663632); Hand1 (e.g., NM_{—}004821 and NP_{—}004812, Hdac1 (e.g., NM_{—}004964 and NP_{—}004955); Hdac2 (e.g., NM_{—}001527.2 and NP_{—}001518.2); HesX1 (e.g., NM_{—}003865 and NP_{—}003856); Hic-5 (e.g., NM_{—}001042454, NM_{—}015927, NP_{—}001035919, and NP_{—}057011); HoxA10 (e.g., NM_{—}018951.3 and NP_{—}061824.3); HoxA11 (e.g., NM_{—}005523.5 and NP_{—}005514.1); HoxB1 (e.g., NM_{—}002144 and NP_{—}002135); HP1a (e.g., NM_{—}001127322, NM_{—}001127321, NM_{—}012117, NP_{—}001120794, NP_{—}001120793, and NP_{—}036249); HP1α (e.g., NM_{—}006807, NM_{—}001127228, NP_{—}001120700, and NP_{—}006798); Irx2 (e.g., NM_{—}001134222, NM_{—}033267, NP_{—}150366, and NP_{—}001127694); Isl1 (e.g., NM_{—}002202 and NP_{—}002193); Jarid2 (e.g., NM_{—}004973 and NP_{—}004964); Jmjd1a (e.g., NM_{—}001146688, NM_{—}018433, NP_{—}001140160, and NP_{—}060903); Jmjd2c (e.g., NM_{—}001146696, NM_{—}001146695, NM_{—}001146694, NM_{—}01506, NP_{—}001140168, NP_{—}001140167, NP_{—}001140166, and NP_{—}055876); Klf-3 (e.g., NM_{—}016531 and NP_{—}057615); Klf-4 (e.g., NM_{—}004235 and NP_{—}004226); Klf-5 (e.g., NM_{—}001730 and NP_{—}001721); Lef1 (e.g., NM_{—}001130714, NM_{—}001130713, NM_{—}016269, NP_{—}001124186, NP_{—}001124185, NP_{—}057353); Lefty-1 (e.g., NM_{—}020997 and NP_{—}066277); Lefty-2 (e.g.,NM_{—}003240 and NP_{—}003231); Lif (e.g., NM_{—}002309 and NP_{—}002300); Lin-28 (e.g., NM_{—}024674 and NP_{—}078950); Mad1 (e.g., NM_{—}001013837, NM_{—}001013836, NM_{—}003550, NP_{—}001013859, NP_{—}001013858, and NP_{—}003541); Mad3 (e.g., NM_{—}001142935, NM_{—}031300, NP_{—}001136407, and NP_{—}112590); Mad4 (e.g., NM_{—}006454 and NP_{—}006445); Mafa (e.g., NM_{—}201589 and NP_{—}963883); Mbd3 (e.g., NM_{—}003926 and NP_{—}003917); Meis1 (e.g., NM_{—}002398 and NP_{—}002389); MeI-18 (e.g., NM_{—}007144 and NP_{—}009075); Meox2 (e.g., NM_{—}005924 and NP_{—}005915); Mta1 (e.g., NM_{—}004689 and NP_{—}004680); Mxi1 (e.g., NM_{—}001008541, NM_{—}005962, NM_{—}130439, NP_{—}001008541, NP_{—}005953, and NP_{—}569157); Myf5 (e.g., NM_{—}005593 and NP_{—}005584); Myst3 (e.g., NM_{—}001099413, NM_{—}006766, NM_{—}001099412, NP_{—}001092883, NP_{—}006757, and NP_{—}001092882); Nac1 (e.g., NM_{—}052876, and NP_{—}443108); Nanog (e.g., NM_{—}024865 and NP_{—}079141); Neurog2 (e.g., NM_{—}024019 and NP_{—}076924); Ngn3 (e.g., NM_{—}020999 and NP_{—}066279); Nkx2.2 (e.g., NM_{—}002509 and NP_{—}002500); Nodal (e.g., NM_{—}018055 and NP_{—}060525); Oct-4 (e.g., NM_{—}203289, NM_{—}002701, NP_{—}976034, and NP_{—}002692); Olig2 (e.g., NM_{—}005806 and NP_{—}005797); Onecut (e.g., NM_{—}004852 and NP_{—}004843); Otx1 (e.g., NM_{—}014562 and NP_{—}055377); Otx2 (e.g., NM_{—}172337, NM_{—}021728, NP_{—}758840, and NP_{—}068374); Pax5 (e.g., NM_{—}016734 and NP_{—}057953); Pax6 (e.g., NM_{—}001127612, NM_{—}001604, NM_{—}000280, NP_{—}001121084, NP_{—}001595, NP_{—}000271); Pdx1 (e.g., NM_{—}000209 and NP_{—}000200); Pias1 (e.g., NM_{—}016166 and NP_{—}057250); Pias2 (e.g., NM_{—}173206, NM_{—}004671, NP_{—}004662, and NP_{—}775298); Pias3 (e.g., NM_{—}006099 and NP_{—}006090); Piasy (e.g., NM_{—}015897 and NP_{—}056981); REST (e.g., NM_{—}005612 and NP_{—}005603); Rex-1 (e.g., NM_{—}174900 and NP_{—}777560); Rfx4 (e.g., NM_{—}213594, NM_{—}002920, NM_{—}032491, NP_{—}998759, NP_{—}115880, and NP_{—}002911); Rif1 (e.g., NM_{—}018151 and NP_{—}060621); Rnf2 (e.g., NM_{—}007212, NP_{—}009143); Rybp (e.g., NM_{—}012234 and NP_{—}036366); Sall4 (e.g., NM_{—}020436.3 and NP_{—}065169.1); Sall2 (e.g., NM_{—}005407.1 and NP_{—}005398.1); Sall1 (e.g., NM_{—}001127892.1, NM_{—}002968.2, NP_{—}001121364.1, NP_{—}002959.2); Scf (e.g., NP_{—}003985, NP_{—}000890, NM_{—}003994, and NM_{—}000899); Scgf (e.g., NM_{—}002975 and NP_{—}002966); Set (e.g., NM_{—}001122821, NM_{—}003011, NP_{—}001116293, and NP_{—}003002); Sip1 (e.g., NM_{—}001009183, NM_{—}001009182, NM_{—}003616, NP_{—}001009183, NP_{—}001009182, NP_{—}003607); Skil (e.g., NM_{—}001145098, NM_{—}001145097, NM_{—}005414, NP_{—}001138570, NP_{—}001138569, and NP_{—}005405); Smarcad1 (e.g., NM_{—}001128430, NM_{—}020159, NM_{—}001128429, NP_{—}001121902, NP_{—}064544, and NP_{—}001121901), Sox-15 (e.g., NM_{—}006942 and NP_{—}008873); Sox-2 (e.g., NM_{—}003106 and NP_{—}003097); Sox-6 (e.g., NM_{—}001145819, NM_{—}001145811, NM_{—}017508, NM_{—}033326, NP_{—}001139291, NP_{—}001139283, NP_{—}201583, and NP_{—}059978); Ssea-1 (e.g., NM_{—}002033 and NP_{—}002024); Stat3 (e.g., NM_{—}213662, NM_{—}003150, NM_{—}139276, NP_{—}998827, NP_{—}644805, and NP_{—}003141); Stella (e.g., NM_{—}199286 and NP_{—}954980); Tbx3 (e.g., NM_{—}016569, NM_{—}005996, NP_{—}057653, and NP_{—}005987); Tcf1 (e.g., NM_{—}000545 and NP_{—}000536); Tcf2 (e.g., NM_{—}000458 and NP_{—}000449); Tcf3 (e.g., NM_{—}001136139, NM_{—}003200, NP_{—}001129611, and NP_{—}003191); Tcf4 (e.g., NM_{—}003199, NM_{—}001083962, NP_{—}001077431, and NP_{—}003190); Tcf7 (e.g., NM_{—}201632, NM_{—}003202, NM_{—}213648, NM_{—}201634, NM_{—}001134852, NM_{—}001134851, NM 201633, NP_{—}001128324, NP_{—}001128323, NP_{—}998813, NP_{—}963965, NP_{—}003193, NP_{—}963963, and NP_{—}963964); Tcf711 (e.g., NM_{—}031283 and NP_{—}112573); Tcl1 (e.g., NM_{—}001098725, NM_{—}021966, NP_{—}001092195, and NP_{—}068801); Tdgf-1 (e.g., NM_{—}003212 and NP_{—}003203); Terf (e.g., NM_{—}001134855, NM_{—}001024941, NM_{—}001024940m NM_{—}016102m NPm01128327, NP_{—}001020112, NP_{—}001020111, and NP_{—}057186); hTert (e.g., NM_{—}198253, NM_{—}198255, NP_{—}937983, NP_{—}937986); Tif1 (e.g., NM_{—}015905, NM_{—}003852, NP_{—}056989, and NP_{—}003843); Tra-1-60 (e.g., NM_{—}001018111, NM_{—}005397, NP_{—}005388, and NP_{—}001018121); Utf-1 (e.g., NM_{—}003577 and NP_{—}003568); Wnt3a (e.g., NM_{—}033131 and NP_{—}149122); Wnt8a (e.g., NM_{—}058244 and NP_{—}490645); YY1 (e.g., NM_{—}003403 and NP_{—}003394); Zeb2 (e.g., NM_{—}014795 and NP_{—}055610); Zfp57 (e.g., NM_{—}001109809 and NP_{—}001103279); Zic3 (e.g., NM_{—}003413 and NP_{—}003404); B-catenin (e.g., NM_{—}001098209, NM_{—}001904, NM_{—}001098210, NP_{—}001091679, NP_{—}001091680, and NP_{—}001895); Coup-Tf2 (e.g., NM_{—}009697, NM_{—}183261, NP_{—}899084, and NP_{—}033827); Zfp281 (e.g., NM_{—}001160251, NM_{—}177643, NP_{—}001153723, and NP_{—}808311); HPV16 E6 (e.g., NP_{—}041325); and HPV16 E7 (e.g., NP_{—}041326), all of which are herein incorporated by reference in their entirety.

### VI. Small Molecule Reprogramming Agents

In preferred embodiments, compositions and methods that are used to increase the pluripotency of a cell include artificial transcription factors (e.g., fusion polypeptides) and one or more small molecule reprogramming agents. In one embodiment, a cell is contacted with at least one artificial pluripotency transcription factor and at least one small molecule reprogramming agent under conditions and for a time sufficient to increase the potency of the cell.

In another embodiment, a cell is contacted with at least one artificial pluripotency transcription factor and at least two, at least three, at least four, at least five, at least six, at least seven, at least eight, at least nine, or at least ten small molecule reprogramming agents under conditions and for a time sufficient to increase the potency of the cell. The increase in potency is objectively measured using the criteria set forth above for assaying the pluripotency characteristics of a cell.

The terms “small molecule reprogramming agent” or “small molecule reprogramming compound” are used interchangeably herein and refer to small molecules that can increase developmental potency of a cell. A “small molecule” refers to an agent that has a molecular weight of less than about 5 kD, less than about 4 kD, less than about 3 kD, less than about 2 kD, less than about 1 kD, or less than about 0.5 kD. Small molecules can be nucleic acids, peptidomimetics, peptoids, carbohydrates, lipids or other organic or inorganic molecules. Libraries of chemical and/or biological mixtures, such as fungal, bacterial, or algal extracts, are known in the art and can be screened with any of the assays of the invention.

In particular embodiments, the small molecule reprogramming agent used herein has a molecular weight of less than 10,000 daltons, for example, less than 8000, 6000, 4000, 2000 daltons, e.g., between 50-1500, 500-1500, 200-2000, 500-5000 daltons. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994). Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990).

The invention disclosed herein encompasses the use of different libraries for the identification of small molecule modulators of one or more components of a cellular pathway associated with cell potency. Libraries useful for the purposes of the invention include, but are not limited to, (1) chemical libraries, (2) natural product libraries, and (3) combinatorial libraries comprised of random peptides, oligonucleotides and/or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see, Cane, D. E., et al., (1998) Science 282:63-68. Combinatorial libraries are composed of large numbers of peptides, oligonucleotides or organic compounds as a mixture. They are relatively easy to prepare by traditional automated synthesis methods, PCR, cloning or proprietary synthetic methods. Of particular interest are peptide and oligonucleotide combinatorial libraries.

Exemplary small molecules suitable for use in the compositions and methods of the present invention include, but are not limited to, IBMV, TSA, VPA, SB203580, Hh-Ag1.3, cyclopamine, valproic acid, purmorphamine, forskolin, TWS119, BIO, cardigiol C, reversine, rosiglitasone, PD98059, WHI-P131, DAPT, 5-aza-C, all-trans RA, and ascorbic acid (Vitamin C), and the like, as described elsewhere herein.

The present invention also provides mixtures of mammalian cells and at least one (e.g., one, two, three, four or more) of: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGF receptor/ALK5 inhibitor, an HDAC inhibitor; an Erk inhibitor, a ROCK inhibitor, and an FGFR inhibitor in any number, amount, or combination.

A. Histone 3 Lysine 9 (H3K9) Methylation

Methylation at H3K9 is implicated in the silencing of euchromatic genes as well as forming silent heterochromatin mentioned. Transcriptional repression involves the recruitment of methylating enzymes and HP1 to the promoter of repressed genes. Delivery of these components of methylation-based silencing is mediated by corepressors such as RB and KAP1. Links between histone methylation and DNA methylation have been demonstrated in Neurospora crassa and in plants, and experimental evidence has shown that histone methylation may be a prerequisite for DNA methylation and transcriptional silencing in Neurospora and Arabidopsis. There are also reports that DNA methylation may trigger H3-K9 methylation in Arabidopsis, suggesting interplay between histone and DNA methylation in maintaining the silent status of the chromatin.

H3K9 methyltransferases and their substrates, include, but are not limited to: SUV39H1; SUV39H2; G9a; ESET/SETDB1; EuHMTase/GLP; CLL8; SpClr4; and RIZ1.

Thus, small molecule reprogramming agents provided herein include inhibitors of H3K9 activity of SUV39H1; SUV39H2; G9a; ESET/SETDB1; EuHMTase/GLP; CLL8; SpClr4; and RIZ1. Exemplary H3K9 inhibitors include, but are not limited to: BIX01294 (see, e.g., Kubicek, et al., 2007), or salts, hydrates, isoforms, racemates, solvates and prodrug forms thereof; SAM analogs, such as, for example methylthio-adenosine (MTA), sinefungin, and S-adenosyl-homocysteine (SAH); inhibitory RNA agents, such as siRNAs, shRNAs, and miRNAs directed against an H3K9 histonemethyltransferase.

In other embodiments, small molecule reprogramming agents provided herein include activators of H3K9 demethylation. Exemplary H3K9 demethylases include, but are not limited to: JHDM2a; JHDM2b; JMJD2A/JHDM3A; JMJD2B; JMJD2C/GASC1; and JMJD2D.

B. Histone 3 Lysine 4 (H3K4) Demethylation

LSD1 acts to demethylate H3K4 and repress transcription (Shi et al., 2004). It is clear that these HDMs will antagonize methylation by being delivered to the right place at the right time (Yamane et al., 2006). Also, the activity of the enzymes are under the influence of the proteins they bind, as in the case of LSD1/BHC110, which acts on nucleosomal substrates in the presence of CoREST (Lee et al., 2005). A very important part of the specificity of these new demethylases also comes down to the state of methylation they act on. Their selectivity for mono-, di-, or trimethylated lysines allows for a larger functional control of lysine methylation (Shi et al., 2007).

Exemplary LSD1 inhibitors include, but are not limited to: nardil (phenelzine sulfate), phenelzin, parnate (tranylcypromine sulfate), tranylcypromine, isocarbazid, selegiline, deprenyl, chlorgyline, pargyline, furazolidon, marplan (isocarboxazid), 1-deprenyl (Eldepryl), moclobemide (Aurorex or Manerix), furazolidone, harmine, harmaline, tetrahydroharmine, nialamide, trans-2-phenyl cyclopropylamine, and the like. Further small molecule reprogramming agents include inhibitory RNA agents, such as siRNAs, shRNAs, and miRNAs directed against an H3K4 histonedemethylase, such as LSD1.

In other embodiments, small molecule reprogramming agents provided herein include activators of H3K4 methylation. Exemplary H3K4 histone methyltransferases include, but are not limited to: MLL1; MLL2; MLL3; MLL4; MLL5; SET1A; SET1B; ASH1; and Sc/Sp SET1.

C. Histone Acetyltransferases (HAT)

Histone acetylation is almost invariably associated with activation of transcription. Acetyltransferases are divided into three main families, GNAT, MYST, and CBP/p300 (Sterner et al., 2000). In general, these enzymes modify more than one lysine but some limited specificity can be detected for some enzymes. Most of the acetylation sites characterized to date fall within the N-terminal tail of the histones, which are more accessible for modification. However, a lysine within the core domain of H3 (K56) has recently been found to be acetylated. A yeast protein, SPT10, may be mediating acetylation of H3K56 at the promoters of histone genes to regulate gene expression (Xu et al., 2005), whereas the Rtt109 acetyltransferase mediates this modification more globally (Han et al., 2007, Driscoll et al., 2007, Schneider et al., 2006). The K56 residue is facing toward the major groove of the DNA within the nucleosome, so it is in a particularly good position to affect histone/DNA interactions when acetylated.

Histones and transcription factors such as p53, E2F1, and GATA1 are known to be substrates for HATs. (The Cancer Journal, 13, 1, 2007, 23). Other non-histone HAT substrates include, for example, Sin 1p, HMG-17, EKLF, TFIIEbeta, and TFIIF.

Exemplary small molecule reprogramming agents include agents that stimulate the expression or activity of HATs such as HAT1, CBP/p300, PCAF/GCN5, TIP60, and HB01, as well as the HATs themselves or active fragments thereof.

D. Histone Deacetylases (HDAC)

The reversal of histone acetylation correlates with transcriptional repression. HDAC inhibitors can induce an open chromatin conformation through the accumulation of acetylated histones, facilitating the transcription of numerous regulatory genes. There are 4 classes of HDAC enzymes. Class I, II, and IV share sequence and structural homology within their catalytic domains and share a related catalytic mechanism that does not require a co-factor, but does require a zinc (Zn) metal ion. In contrast, class III (sirtuins) do not share sequence or structural homology with the other HDAC families and use a distinct catalytic mechanism that is dependant on the oxidized form of nicotinamide adenine dinucleotide (NAD+) as a co-factor. Sirtuins have been linked to counteracting age associated diseases such as type II diabetes, obesity and neurodegenerative diseases (Oncogene, 2007, 26, 5528).

Illustrative examples of HDAC inhibitors include, for example, butyrate; suberoylanilide hydroxamic acid (SAHA, a.k.a. Vorinostat); Belinostat/PXD101; MS275; LAQ824/LBH589; CI994; MGCD0103; nicotinamide, as well derivatives of NAD, dihydrocoumarin, naphthopyranone, and 2-hydroxynaphaldehydes; Trichostatin A; Chlamydocin; cyclic tetrapeptide trapoxin A and trapoxin B; electrophilic ketones; aliphatic acid compounds such as phenylbutyrate and valproic acid; and the natural product Apicidin, among others.

E. L-Type Calcium Channel Agonists

Exemplary L-type calcium channel agonists include, but are not limited to, BayK8644, Dehydrodidemnin B, FPL 64176, S(+)-PN 202-791, and CGP 48506, among others.

F. cAMP Activators

Exemplary activators of the cAMP pathway include, but are not limited to, forskolin, FSH, milrinone, cilostamide, rolipram, dbcAMP, and 8-Br-cAMP, among others.

G. DNA Methyltransferase Inhibitors

Exemplary DNA methyltransferase (DNMT) inhibitors include, but are not limited to antibodies that bind DNMT, dominant negative DNMT variants, and siRNA and antisense nucleic acids that suppress expression of DNMT. DNMT inhibitors include, but are not limited to, RG108, 5-aza-C (5-azacitidine or azacitidine), 5-aza-2′-deoxycytidine (5-aza-CdR), decitabine, doxorubicin, EGCG ((−)-epigallocatechin-3-gallate), zebularine, procainamide, 5,6-dihydro-5-azacytidine, procaine 5-fluoroouracil, procaine hydrochloride, epigallocatechin-3-gallate (EFOG),psammaplin A, and MG98, among others.

H. Nuclear Receptor Ligands

Exemplary nuclear receptor ligands, include, but are not limited to: dexamethasone, ciglitazone, Fmoc-Leu, Bexarotene, estradiol, all-trans retinoic acid, 13-cis retinoic acid, dexamethasone, clobetasol, androgens, thyroxine, vitamin D3 glitazones, troglitazone, pioglitazone, rosiglitazone, prostaglandins, and fibrates (e.g., bezafibrate, ciprofibrate, gemfibrozil, fenofibrate and clofibrate) and any synthetic analogs thereof.

I. GSK-3β Inhibitors

Inhibitors of GSK-3β include, but are not limited to antibodies that bind GSK-3β, dominant negative GSK-3β variants, and siRNA and antisense nucleic acids that target GSK-3β. Exemplary GSK-3β inhibitors include, but are not limited to, Kenpaullone, I-Azakenpaullone, CHIR99021, CHIR98014, AR-A014418, CT 99021, CT 20026, SB216763, AR-A014418, lithium, SB 415286, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(1-pyridyl)-[1,3,4]-oxadiazole, OTDZT, alpha-4-Dibromoacetophenone, R-AO 144-18, 3-(1-(3-Hydroxypropyl)-1H-pyrrolo[2,3-b]pyridin-3-yl]-4-pyrazin-2-yl-pyrrole-2,5-dione; TWSI 19 pyrrolopyrimidine compound, L803 H-KEAPPAPPQSpP-NH2 or its myristoylated form; 2-Chloro-1-(4,5-dibromo-thiophen-2-yl)-ethanone, SB216763, and SB415286.

J. MEK Inhibitors

Exemplary MEK inhibitors include, but are not limited to antibodies to MEK, dominant negative MEK variants, and siRNA and antisense nucleic acids that suppress expression of MEK. Other exemplary MEK inhibitors include, but are not limited to, PD0325901, PD98059, UO126, SL327, ARRY-162, PD184161, PD184352, sunitinib, sorafenib, Vandetanib, pazopanib, Axitinib, GSKI 120212, ARRY-438162, RO5126766, XL518, AZD8330, RDEAI 19, AZD6244, and PTK787.

Additional MEK inhibitors include those compounds disclosed in International Published Patent Applications WO 99/01426, WO 02/06213, WO 03/077914, WO 05/051301 and WO2007/044084.

Further illustrative examples of MEK inhibitors include the following compounds:—6-(4-Bromo-2-chloro-phenylamino)-7-fluoro-3-methyl-3H-benzoimidazol-e-5-carboxylic acid (2,3-dihydroxy-propoxy)-amide; 6-(4-Bromo-2-chloro-phenylamino)-7-fluoro-3-(tetrahydro-pyran-2-ylm-ethyl)-3H-benzoimidazole-5-carboxylic acid (2-hydroxy-ethoxy)-amide, 1-[6-(4-Bromo-2-chloro-phenylamino)-7-fluoro-3-methyl-3H-benzoimida-zol-5-yl]-2-hydroxy-ethanone, 6-(4-Bromo-2-chloro-phenylamino)-7-fluoro-3-methyl-3H-benzoimidazol-e-5-carboxylic acid (2-hydroxy-1,1-dimethyl-ethoxy)-amide, 6-(4-Bromo-2-chloro-phenylamino)-7-fluoro-3-(tetrahydro-furan-2-ylm-ethyl)-3H-benzoimidazole-5-carboxylic acid (2-hydroxy-ethoxy)-amide, 6-(4-Bromo-2-fluoro-phenylamino)-7-fluoro-3-methyl-3H-benzoimidazol-e-5-carboxylic acid (2-hydroxy-ethoxy)-amide, 6-(2,4-Dichloro-phenylamino)-7-fluoro-3-methyl-3H-benzoimidazole-5-carboxylic acid (2-hydroxy-ethoxy)-amide, 6-(4-Bromo-2-chloro-phenylamino)-7-fluoro-3-methyl-3H-benzoimidazol-e-5-carboxylic acid (2-hydroxy-ethoxy)-amide, referred to hereinafter as MEK inhibitor 1; 2-[(2-fluoro-4-iodophenyl)amino]-N-(2-hydroxyethoxy)-1,5-dimethyl-6-1-oxo-1,6-dihydropyridine-3-carboxamide; referred to hereinafter as MEK inhibitor 2; and 4-(4-bromo-2-fluorophenylamino)-N-(2-hydroxyethoxy)-1,5-dimethyl-6-oxo-1,6-dihydropyridazine-3-carboxamide or a pharmaceutically acceptable salt thereof.

K. TGFβ Receptor/ALK5 Inhibitors

Exemplary ALK5 inhibitors include antibodies to ALK5, dominant negative variants of ALK5, and antisense nucleic acids that suppress expression of ALK5. Exemplary ALK5 inhibitors include, but are not limited to, SB431542, A-83-01, 2-(3-(6-Methylpyridin-2-yl)-1H-pyrazol-4-yl)-1,5-naphthyridine, Wnt3a/BIO, BMP4, GW788388, SM16, IN-1130, GW6604, SB-505124, and pyrimidine derivatives, see, e.g., WO2008/006583, herein incorporated by reference.

Further, while “an ALK5 inhibitor” is not intended to encompass non-specific kinase inhibitors, an “ALK5 inhibitor” should be understood to encompass inhibitors that inhibit ALK4 and/or ALK7 in addition to ALK5, such as, for example, SB-431542 (see, e.g., Inman, et al, J. Mol. Phamacol. 62(1): 65-74 (2002). Without intending to limit the scope of the invention, it is believed that ALK5 inhibitors affect the mesenchymal to epithelial conversion/transition (MET) process. TGFβ/activin pathway is a driver for epithelial to mesenchymal transition (EMT). Therefore, inhibiting the TGFβ/activin pathway can facilitate MET (i.e. reprogramming) process.

In view of the data herein showing the effect of inhibiting ALK5, it is believed that inhibition of the TGFβ/activin pathway will have similar effects. Thus, any inhibitor, e.g., upstream or downstream of the TGFβ/activin pathway can be used in combination with, or instead of, ALK5 inhibitors as described in each paragraph herein. Exemplary TGFβ/activin pathway inhibitors include but are not limited to: TGF receptor inhibitors, inhibitors of SMAD ⅔ phosphorylation, inhibitors of the interaction of SMAD ⅔ and SMAD 4, and activators/agonists of SMAD 6 and SMAD 7. Furthermore, the categorizations described below are merely for organizational purposes and one of skill in the art would know that compounds can affect one or more points within a pathway, and thus compounds may function in more than one of the defined categories.

TGFβ receptor inhibitors can include antibodies to, dominant negative variants of and siRNA or antisense nucleic acids that target TGFβ receptors. Specific examples of inhibitors include but are not limited to SU5416; 2-(5-benzo[1,3]dioxol-5-yl-2-tert-butyl-3H-imidazol-4-yl)-6-methylpyridine hydrochloride (SB-505124); lerdelimumb (CAT-152); metelimumab (CAT-192); GC-1008; IDI 1; AP-12009; AP-11014; LY550410; LY580276; LY364947; LY2109761; SB-505124; SB-431542; SD-208; SM16; NPC-30345; Ki26894; SB-203580; SD-093; Gleevec; 3,5,7,2′,4′-pentahydroxyfiavone (Morin); activin-M108A; P144; soluble TBR2-Fc; and antisense transfected tumor cells that target TGFβ receptors. (See, e.g., Wrzesinski, et al., Clinical Cancer Research 13(18):5262-5270 (2007); Kaminska, et al., Acta Biochimica Polonica 52(2):329-337 (2005); and Chang, et al., Frontiers in Bioscience 12:4393-4401 (2007).

Inhibitors of SMAD ⅔ phosphorylation can include antibodies to, dominant negative variants of and antisense nucleic acids that target SMAD2 or SMAD3. Specific examples of inhibitors include PD169316; SB203580; SB-431542; LY364947; A77-01; and 3,5,7,2′,4′-pentahydroxyflavone (Morin). (See, e.g., Wrzesinski, supra; Kaminska, supra; Shimanuki, et al., Oncogene 26:3311-3320 (2007); and Kataoka, et al, EP1992360, incorporated herein by reference).

Inhibitors of the interaction of SMAD 2/3 and smad4 can include antibodies to, dominant negative variants of and antisense nucleic acids that target SMAD2, SMAD3 and/or smad4. Specific examples of inhibitors of the interaction of SMAD ⅔ and SMAD4 include but are not limited to Trx-SARA, Trx-xFoxHlb and Trx-Lef1. (See, e.g., Cui, et al, Oncogene 24:3864-3874 (2005) and Zhao, et al., Molecular Biology of the Cell, 17:3819-3831 (2006)).

Activators/agonists of SMAD 6 and SMAD 7 include but are not limited to antibodies to, dominant negative variants of and antisense nucleic acids that target SMAD 6 or SMAD 7. Specific examples of inhibitors include but are not limited to smad7-as PTO— oligonucleotides. (See, e.g., Miyazono, et al., U.S. Pat. No. 6,534,476, and Steinbrecher, et al., US2005119203, both incorporated herein by reference).

L. ERK Inhibitors

Exemplary ERK inhibitors include, but are not limited to antibodies to ERK, dominant negative ERK variants, and siRNA and antisense nucleic acids that suppress expression of ERK. Other exemplary ERK inhibitors include PD98059, U0126, FR180204, sunitinib, sorafenib, Vandetanib, pazopanib, Axitinib, and PTK787.

M. ROCK Inhibitors

ROCKs are serine/threonine kinases that serve as target proteins for Rho (of which three isoforms exist—RhoA, RhoB and RhoC). Exemplary ROCK inhibitors include, but are not limited to antibodies to ROCK, dominant negative ROCK variants, and siRNA and antisense nucleic acids that suppress expression of ROCK. Other exemplary ROCK inhibitors include, but are not limited to: Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide.

N. FGFR Inhibitors

Exemplary FGFR inhibitors include, but are not limited to antibodies to FGFR, dominant negative FGFR variants, and siRNA and antisense nucleic acids that suppress expression of FGFR. Exemplary FGFR inhibitors include, but are not limited to RO-4396686, CHIR-258, PD 173074, PD 166866, ENK-834, ENK-835, SU5402, XL-999, SU6668, CHIR-258, R04383596, and BIBF-1120.

In one embodiment, a composition that increases the potency of a cell comprises: (a) at least one APTF comprising: i) one or more DBDs selected from the group consisting of: Oct-3/4, Nanog, Sox2, Klf-4, Stella, and Sall4; and ii) one or more transcriptional activation domains selected from the group consisting of: VP16, VP64, SV40 Large T-antigen, E1A activation domain, relA, and EGFR-1; and (b) one or more small molecule reprogramming agents selected from the group consisting of: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor.

In a particular embodiment, a composition that increases the potency of a cell comprises: (a) at least one APTF comprising a CPP, NLS, DBD, and TAD; and (b) one or more of an agent that inhibits H3K9 methylation or promotes H3K9 demethylation and an L-type Ca channel agonist.

In another particular embodiment, a composition that increases the potency of a cell comprises: (a) at least one APTF comprising a CPP, NLS, DBD, and TAD; and (b) one or more of an agent that inhibits H3K4 demethylation or promotes H3K4 methylation and a GSK3 inhibitor.

In another particular embodiment, a composition that increases the potency of a cell comprises: (a) at least one APTF comprising a CPP, NLS, DBD, and TAD; and (b) one or more small molecule reprogramming agents selected from the group consisting of: an agent that inhibits H3K9 methylation or promotes H3K9 demethylation; an agent that inhibits H3K4 demethylation or promotes H3K4 methylation; a GSK3 inhibitor, a MEK inhibitor, a TGFβ receptor/ALK5 inhibitor; an Erk inhibitor, a ROCK inhibitor, and an FGFR inhibitor.

In another particular embodiment, a composition that increases the potency of a cell comprises: (a) at least one APTF comprising a CPP, NLS, DBD, and TAD; and (b) one or more, two or more, three or more, four or more, five or more, six or more, seven or more, eight or more, nine or more, or ten or more small molecule reprogramming agents selected from the group consisting of: an agent that inhibits H3K9 methylation or promotes H3K9 demethylation; an agent that inhibits H3K4 demethylation or promotes H3K4 methylation; a GSK3 inhibitor, a MEK inhibitor, a TGFβ receptor/ALK5 inhibitor; an Erk inhibitor, a ROCK inhibitor, and an FGFR inhibitor.

In one embodiment, the cell can be first contacted with one or more APTFs and then contacted with a composition comprising one or more small molecule reprogramming agents. In another embodiment, the cell can be first contacted with a composition comprising one or more small molecule reprogramming agents and then contacted with one or more APTFs.

In particular embodiments, incompletely pluripotent human stem cells are contacted with one or more artificial transcription factors in combination with one or more, two or more, three or more, four or more, five or more, six or more, seven or more, eight or more, nine or more, or ten or more small molecule reprogramming agents selected from the group consisting of an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor and thereby increasing the pluripotency of the cell to a more primitive pluripotent state.

In a related embodiment, incompletely pluripotent human stem cells, such are contacted with one or more, two or more, three or more, four or more, five or more, six or more, seven or more, eight or more, nine or more, or ten or more small molecule reprogramming agents selected from the group consisting of an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor and thereby increasing the pluripotency of the cell to a more primitive pluripotent state. In certain embodiments, the incompletely pluripotent cells are hiPSCs or hEpiSCs.

### VII. Formulations and Compositions

The compositions of the invention may comprise one or more polypeptides, polynucleotides, vectors comprising same, etc., as described herein, formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., other proteins, polypeptides, small molecule reprogramming agents or various pharmaceutically-active agents. There is virtually no limit to other components that may also be included in the compositions, provided that the additional agents do not adversely affect the ability of the composition to increase, establish, and/or maintain the pluripotency of a cell.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain applications, the compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, optionally mixing with CPP polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the invention may comprise one or more repressors and/or activators comprised of a combination of any number of polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the invention may be administered in combination with other agents as well, such as, e.g., cells, other proteins or polypeptides or various pharmaceutically-active agents.

In a particular embodiment, a formulation or composition according to the present invention comprises a cell contacted with a combination of any number of polypeptides, polynucleotides, and small molecules, as described herein. In a related embodiment, a formulation or composition according to the present invention comprises a cell contacted with a combination of any number of polypeptides, polynucleotides, and small molecules, as described herein, formulated in a pharmaceutically acceptable cell culture medium.

In certain aspects, the present invention provides formulations or compositions suitable for the delivery of a combination of any number of repressors and/or activators that modulate a component of a cellular potency pathway, as provided herein, to a cell in tissue culture, such as an in vitro or ex vivo cell or population of cells. Exemplary formulations for ex vivo delivery may include the use of viral vector systems (i.e., viral-mediated transduction) including, but not limited to, retroviral (e.g., lentiviral) vectors, adenoviral vectors, adeno-associated viral vectors, and herpes viral vectors, among others.

Exemplary formulations for ex vivo delivery may also include the use of various transfection agents known in the art, such as calcium phosphate, electoporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane. By including a small amount of an anionic lipid in an otherwise cationic liposome the DNA can be incorporated into the internal surface of the liposome, thus protecting it from enzymatic degradation. In certain embodiments, liposome formulations may optimized for a particular target cell type, such as for pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like. To facilitate uptake into the cell as endosomes, certain embodiments may employ targeting proteins in liposomes, including, for example, anti-MHC antibody, transferrin, the Sendai virus or its F protein, in addition to other desirable targeting agents. It is appreciated that Sendai viral proteins allow the plasmid DNA to escape from the endosome into the cytoplasm, thus avoiding degradation. As an additional example, the inclusion of a DNA binding protein (e.g., 28 kDa high mobility group 1 protein) enhances transcription by bringing the plasmid into the nucleus. Certain embodiments may include incorporating the Epstein-Barr virus Ori p and EBNA1 genes in the plasmid to maintain the plasmid as an episomal element.

Certain formulations may employ the use of molecular conjugates, which consist of protein or synthetic ligands to which a DNA binding agent has been attached. Delivery to the cell can be improved by using similar techniques to those for liposomes. Exemplary targeting proteins include asialoglycoprotein, the Vpr protein from HIV, transferrin, polymeric IgA, and adenoviral proteins.

In certain aspects, the present invention provides pharmaceutically acceptable compositions which comprise a therapeutically-effective amount of one or more repressors and/or activators, including, but not limited to nucleic acd-based agents, as described herein, formulated together with one or more pharmaceutically acceptable carriers (additives) and/or diluents (e.g., pharmaceutically acceptable cell culture medium). Methods for the delivery of nucleic acid molecules are described in Akhtar et al., 1992, Trends Cell Bio., 2:139; and Delivery Strategies for Antisense Oligonucleotide Therapeutics, ed. Akhtar; Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres.

### VIII. Methods of Delivery

In one embodiment, cells are contacted with a composition comprising one or more artificial pluripotency transcription factors and/or a combination of small molecule reprogramming agents, wherein the ATPFs and small molecules increase or establish the pluripotency of a cell. It is contemplated that the cells of the invention may be contacted in vitro, ex vivo, or in vivo.

Once formulated, the compositions of the invention can be administered (as proteins/polypeptides, or in the context of expression vectors for gene therapy) directly to the subject or delivered ex vivo, to cells derived from the subject (e.g., as in ex vivo gene therapy). Direct in vivo delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, for example, International PCT Publication No. WO 93/14778. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus (and adeno-associated virus) or alphavirus, all well known in the art.

Illustrative, but non-limiting methods of nucleic acid and polypeptide delivery are further discussed below.

In certain embodiments, it will be preferred to deliver one or more pluripotency factors to a cell using a viral vector or other in vivo polynucleotide delivery technique. In a preferred embodiment, the viral vector is a non-integrating vector or a transposon-based vector. This may be achieved using any of a variety or well-known approaches, several of which are outlined below for purposes of illustration. Exemplary methods of delivery are further described in U.S. Provisional Patent Application No. 61/241,647, the disclosure of which is herein incorporated by reference.

A. Adenovirus Vectors

One illustrative method for in vivo delivery of one or more nucleic acid sequences involves the use of a genetically engineered adenovirus expression vector. Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). Adenoviral infection of host cells does not result in chromosomal integration because adenoviral DNA can replicate in an episomal manner without potential genotoxicity. In addition, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

B. Retrovirus Vectors

The retroviruses are a group of single-stranded RNA viruses characterized by an ability to convert their RNA to double-stranded DNA that stably integrates into cellular chromosomes as a provirus and directs synthesis of viral proteins. Most retrovirus will only infect actively dividing cells; thus, limiting their utility. However, another class of retrovirus, the lentivirus, is able to infect dividing as well as non-dividing cells; thus making lentivirus the vector of choice in virally mediated transgenesis.

Exemplary lentiviral vectors including vectors based on HIV-1, HIV-2 simian immunodeficiency virus (SIV), feline immunodeficiency virus (FIV), equine infectious anemia virus (EIAV), bovine immunodeficiency virus (BIV), Jembrana disease virus (JDV), visna virus (VV), and caprine arthritis encephalitis virus (CAEV). Human immunodeficiency virus type 1 (HIV-1) has long been known to form pseudotypes by the incorporation of heterologous glycoproteins (GPs) through phenotypic mixing, and thus, broadening the tropism of the virus. Exemplary pseudotyping glycoproteins include, but are not limited to glycoproteins derived from the following viruses: Ebola, GALV, JSRV, LCMV, Marburg, Mokola, Rabies, RD114, RRV, SeV F, and VSV (Cronin et al., 2006).

In one embodiment, an APTF is cloned into a lentivirus and is flanked by LoxP, AttL/AttR, or FRT sites. Once the provirus has integrated, the cell can be treated with Cre, Int and X is, or Flp recombinases, respectively, to excise the integrated proviral cassette. However, excision is not complete, leaving behind a single recombinase site in each strategy. In another embodiment, the lentivirus has an inducible promoter, such as a doxycycline inducible promoter.

Recent advances have provided episomal forms of retroviral vectors based on lentiviruses. The nonintegrating lentiviral vectors retain the high transduction efficiency and broad tropism of conventional lentiviruses but avoid the potential problems associated with the nonspecific integration of a transgene. In this respect they are particularly useful from a safety standpoint, and in certain embodiments, are preferred.

C. Adeno-Associated Virus Vectors

AAV (Ridgeway, 1988; Hermonat & Muzycska, 1984) is a parovirus, discovered as a contamination of adenoviral stocks. AAV is a good choice of delivery vehicle due to its safety, i.e., genetically engineered (recombinant) AAV does not integrate into the host genome. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

D. Other Viral Vectors as Expression Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), polioviruses and herpes viruses may be employed. They offer several attractive features for various mammalian cells (Friedmann, 1989; Ridgeway, 1988; Coupar et al., 1988; Horwich et al., 1990).

E. Non-Viral Methods

piggyBac (PB) transposition is a seamless and reversible platform to genetically alter cells. A transgene such as polynucleotide encoding an artificial pluripotency transcription factor is flanked by inverted terminal repeats in a PB transposon. The PB transposon is introduced into a cell along with an inducible transpose to catalyze the insertion of the PB transposon. Once inserted into the genome the APTFs or other transgene inserts are expressed. When expression of the APTFs or other transgene is no longer required, transient expression of a transpose allows for seamless excision of the transposon from the genome. Although the genome was genetically altered, the genetic alteration was reversible. Thus, this method represents the safest method of genetic engineering. The piggyBac transposon system has been used to generate iPSCs (Woltjen et al., 2009; Guo et al., 2009).

In another embodiment, APTFs comprising one or more CPP polypeptides are incubated in stem cell growth medium with cells. The incubation step is repeated one, two, three, four, or five or more times in order to provide a continuous supply of the APTFs to the cell. The APTFs translocate to the nucleus of the cell and increase transcription of pluripotency genes; thereby increasing the potency of the cell. Somatic cell reprogramming of mouse and human fibroblasts using pluripotency proteins has been demonstrated (Zhou et al., 2009 and Kim et al., 2009, respectively).

In one embodiment, a polynucleotide may be administered directly to a cell via microinjection. Dubensky et al., (1984) successfully injected polyomavirus DNA in the form of calcium phosphate precipitates into liver and spleen of adult and newborn mice demonstrating active viral replication and acute infection. Benvenisty & Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In another embodiment, polynucleotides are administered to cells via electroporation.

In related embodiments, liposomes act as gene and or polypeptide delivery vehicles and are described in U.S. Pat. No. 5,422,120; WO 95/13796; WO 94/23697; WO 91/14445; and EP 0524968. Additional approaches are described in Philip, Mol. Cell. Biol. 14:2411 (1994), and in Woffendin, Proc. Natl. Acad. Sci. (1994) 91:11581-11585. The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents.

For use with the methods and compositions disclosed herein, liposomes typically comprise a polypeptide or fusion polypeptide as disclosed herein, a lipid component, e.g., a neutral and/or cationic lipid, and optionally include a receptor-recognition molecule such as an antibody that binds to a predetermined cell surface receptor or ligand (e.g., an antigen). A variety of methods are available for preparing liposomes as described in, e.g.; U.S. Pat. Nos. 4,186,183; 4,217,344; 4,235,871; 4,261,975; 4,485,054; 4,501,728; 4,774,085; 4,837,028; 4,235,871; 4,261,975; 4,485,054; 4,501,728; 4,774,085; 4,837,028; 4,946,787; PCT Publication No. WO 91/17424; Szoka et al. (1980) Ann. Rev. Biophys. Bioeng. 9:467; Deamer et al. (1976) Biochim. Biophys. Acta 443:629-634; Fraley, et al. (1979) Proc. Natl. Acad. Sci. USA 76:3348-3352; Hope et al. (1985) Biochim. Biophys. Acta 812:55-65; Mayer et al. (1986) Biochim. Biophys. Acta 858:161-168; Williams et al. (1988) Proc. Natl. Acad. Sci. USA 85:242-246; Liposomes, Ostro (ed.), 1983, Chapter 1); Hope et al. (1986) Chem. Phys. Lip. 40:89; Gregoriadis, Liposome Technology (1984) and Lasic, Liposomes: from Physics to Applications (1993). Suitable methods include, for example, Sonication, extrusion, high pressure/homogenization, microfluidization, detergent dialysis, calcium-induced fusion of small liposome vesicles and ether-fusion methods, all of which are well known in the art.

In certain embodiments, it may be desirable to target a liposome using targeting moieties that are specific to a particular cell type, tissue, and the like. Targeting of liposomes using a variety of targeting moieties (e.g., ligands, receptors, and monoclonal antibodies) has been previously described. See, e.g., U.S. Pat. Nos. 4,957,773 and 4,603,044. Standard methods for coupling targeting agents to liposomes are used. These methods generally involve the incorporation into liposomes of lipid components, e.g., phosphatidylethanolamine, which can be activated for attachment of targeting agents, or incorporation of derivatized lipophilic compounds, such as lipid derivatized bleomycin. Antibody targeted liposomes can be constructed using, for instance, liposomes which incorporate protein A. See Renneisen et al. (1990) J. Biol. Chem. 265:16337-16342 and Leonetti et al. (1990) Proc. Natl. Acad. Sci. USA 87:2448-2451.

### IX. Implants

In various illustrative embodiments, the invention provides cell-based compositions that can be employed as cell-based therapies in mammals, for example, in the repair, regeneration, or replacement of a cell, tissue, or organ. Generally, such methods involve providing a cell-based composition to a desired site in an individual, such as an in vivo cell, tissue, organ, or an implant comprising a biocompatible matrix implanted in vivo.

As used herein, the term “implant” refers to a biocompatible natural and/or synthetic structure comprising one or more cell-based compositions, cells, tissues, polymers, polynucleotides, magnetic particles, agarose particles, plastic particles, polypeptides, oligosaccharides, lipids, small molecules, lattices, and/or matrices that is injected or engrafted within a patient or subject that is suitable for directing or attracting a cell-based composition to repair, regenerate, or replace a cell, tissue or organ in vivo. In various embodiments, an implant refers to a matrix, as defined herein, that is suitable for directing or attracting a cell-based composition to repair, regenerate, or replace a cell, tissue or organ in vivo in a patient.

As used herein, the term “matrix” refers to a biocompatible natural and/or synthetic environment that is suitable for directing or attracting a cell-based composition to repair, regenerate, or replace a cell, tissue or organ in vivo. Components of a natural or synthetic matrix, include but are not limited to, any number or combination of cells, tissues, polymers, polynucleotides, magnetic particles, agarose particles, plastic particles, polypeptides, oligosaccharides, lipids, or small molecules.

It would be appreciated by one having skill in the art that an implant can comprise any of the matrices described herein, with any additional components or added features as described herein. Exemplary implants are described in U.S. Provisional Patent Application No. 61/241,647, the disclosure of which is herein incorporated by reference.

### X. Cell Cultures and Cell Culture Compositions

In various embodiments, the compositions and methods of the present invention comprise the culture of cells with compositions of the present invention.

A. Mouse Embryonic Stem Cell Culture

Mitotically inactivated cell feeder layers were first used to support difficult-to-culture epithelial cells (Puck et al., 1956) and were later successfully adapted for the culture of mouse EC cells (Martin and Evans 1975) and mouse ESCs (Evans and Kaufman 1981). Medium that is “conditioned” by coculture with various cells was found to be able to sustain ESCs in the absence of feeders, and fractionation of conditioned medium led to the identification of leukemia inhibitory factor (LIF), a cytokine that sustains ESCs (Smith et al., 1988; Williams et al., 1988). LIF and its related cytokines act via the gp130 receptor (Yoshida et al., 1994). Binding of LIF induces dimerization of LIFR/gp130 receptors, which in turn activates the Janus-associated tyrosine kinases (JAK)/the latent signal transducer and activator of transcription factor (STAT3) (Yoshida et al., 1994), and Shp2/ERK mitogen-activated protein kinase (MAPK) cascade (Takahashi-Tezuka et al., 1998). STAT3 activation alone is sufficient for LIF-mediated self-renewal of mouse ESCs in the presence of serum (Matsuda et al., 1999). Activation of ERK, however, appears to impair mouse ESC proliferation. In contrast, suppression of the ERK pathway by the addition of MEK inhibitor PD098059 promotes ESC self-renewal (Burdon et al., 1999). Thus, the proliferative effect of LIF on mouse ESCs requires a finely tuned balance between positive and negative effectors.

B. Human Embryonic Stem Cell Culture

The specific factors used to sustain mouse ESCs do not support traditional human ESCs such as the hESC line H1. In contrast to mESC culture conditions, traditional hESCs cultured in medium containing LIF and components of the BMP pathway (e.g., BMP4) cause hESCs to differentiate into trophoblasts or primitive endoderm. In further contrast to mESC culture conditions, while hESCs require bFGF and Activin signaling to maintain pluripotency, mESCs differentiate if cultured in bFGF and Activin.

C. Human iPSC Culture

Li and colleagues, 2009, have determined that hiPSCs can be cultured in mESC culture conditions supplemented with inhibitors of ALK5, GSK3, and MEK. Specifically, Li et al. reprogrammed human fibroblasts using viral Oct-3/4, Sox-2, Klf-4, and cMyc in mESC culture medium comprising hLIF. The transduced cells were subsequently cultured in mESC medium containing LIF and A-83-01 (e.g., ALK 5 inhibitor), CHIR99021 (e.g., GSK3 inihibitor), and PD0325901 (e.g., MEK inhibitor). The hiPSCs cultured in this manner form compact, domed, ALP positive colonies that closely resemble mESCs including expression of the pluripotency markers Oct4, Sox2, Nanog, Rex-1, TDGF2, and FGF4. Moreover, the iPSCs are able to form embryoid bodies and teratomas comprising tissues from each of the germ layers. Importantly, collateral experiments were conducted with riPSCs which had not previously been shown to contribute to rat chimeras. Modifying the riPSCs culture to reflect mESC culture conditions in combination with ALK, ERK, and GSK3 inhibitors, allowed the resultant riPSCs to reasonably contribute to rat chimeras.

Without wishing to be bound to any particular theory, hiPSCs that are cultured in the traditional manner may not reprogram hiPSCs to the most primitive pluripotent state, that is, the state of pluripotency with the most developmental potency. However, by culturing hiPSCs in mESC culture conditions, in combination with small molecular reprogramming agents, such as inhibitors of ALK5, MEK, and GSK3, one can achieve a more primitive hiPSC with the most developmental potency and plasticity.

The practice of the present invention will employ, unless indicated specifically to the contrary, conventional methods of chemistry, biochemistry, organic chemistry, molecular biology, microbiology, recombinant DNA techniques, genetics, immunology, cell biology, stem cell protocols, cell culture and transgenic biology that are within the skill of the art, many of which are described below for the purpose of illustration. Such techniques are explained fully in the literature. See, e.g., Sambrook, et al., Molecular Cloning: A Laboratory Manual (3^{rd} Edition, 2001); Sambrook, et al., Molecular Cloning: A Laboratory Manual (2^{nd} Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-interscience; Glover, DNA Cloning: A Practical Approach, vol. I & II (IRL Press, Oxford, 1985); Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Fire et al., RNA Interference Technology: From Basic Science to Drug Development (Cambridge University Press, Cambridge, 2005); Schepers, RNA Interference in Practice (Wiley-VCH, 2005); Engelke, RNA Interference (RNAi): The Nuts & Bolts of siRNA Technology (DNA Press, 2003); Gott, RNA Interference, Editing, and Modification: Methods and Protocols (Methods in Molecular Biology; Human Press, Totowa, N.J., 2004); Sohail, Gene Silencing by RNA Interference: Technology and Application (CRC, 2004); Clarke and Sanseau, microRNA: Biology, Function & Expression (Nuts & Bolts series; DNA Press, 2006); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008); Hogan et al., Methods of Manipulating the Mouse Embyro (2^{nd} Edition, 1994); Nagy et al., Methods of Manipulating the Mouse Embryo (3^{rd} Edition, 2002), and The zebrafish book. A guide for the laboratory use of zebrafish (Danio rerio), 4th Ed., (Univ. of Oregon Press, Eugene, Oreg., 2000).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of increasing the potency of a cell, comprising contacting the cell with one or more polynucleotides, each polynucleotide comprising an artificial pluripotency transcription factor (APTF), wherein the APTF comprises polypeptide domains encoding:
a) a nuclear localization sequence (NLS);
b) a DNA binding domain (DBD); and
c) a transcriptional activation domain (TAD);
wherein at least two of the polypeptide domains of a)-c) are heterologous polypeptide domains, and
wherein said contacting is performed under conditions and for a time sufficient to induce at least one pluripotent stem cell characteristic in the cell, thereby increasing the potency of the cell.

2. The method of claim 1, wherein the APTF comprises a cell permeable peptide (CPP).

3.-4. (canceled)

5. The method of claim 1, wherein the DBD is selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

6.-8. (canceled)

9. The method of claim 1, wherein the TAD is selected from the group consisting of: VP16, VP64, SV40 Large T-antigen, E1A activation domain, relA, and EGFR-1.

10. The method of claim 1, further comprising contacting the cell with one or more small molecule reprogramming agents selected from the group consisting of:
an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor.

11.-13. (canceled)

14. The method of claim 10, comprising contacting the cell in a culture medium containing hLIF, an ALK5 inhibitor, a MEK inhibitor, and a GSK3 inhibitor.

15. A method of increasing the potency of a cell, comprising contacting the cell with one or more artificial pluripotency transcription factor polypeptides, wherein each APTF polypeptide comprises polypeptide domains encoding:
a) a nuclear localization sequence (NLS);
b) a DNA binding domain (DBD); and
c) a transcriptional activation domain (TAD);
wherein at least two of the polypeptide domains of a)-c) are heterologous polypeptide domains, and
wherein said contacting is performed under conditions and for a time sufficient, to induce at least one pluripotent stem cell characteristic in the cell, thereby increasing the potency of the cell.

16. The method of claim 15, wherein the APTF comprises a CPP.

17. The method of claim 15, wherein the DBD is selected from the group consisting of: Oct-3/4, Cdx-2, Gbx2, Gsh1, HesX1, HoxA10, HoxA11, HoxB1, Irx2, Is11, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

18.-20. (canceled)

21. The method of claim 15, wherein the TAD is selected from the group consisting of: VP16, VP64, SV40 Large T-antigen, E1A activation domain, relA, and EGFR-1.

22. The method of claim 15, further comprising contacting the cell with one or more small molecule reprogramming agents selected from the group consisting of: an agent that inhibits H3K9 methylation or promotes H3K9 demethylation; an agent that inhibits H3K4 demethylation or promotes H3K4 methylation; an agent that inhibits histone deacetylation or promotes histone acetylation; an L-type Ca channel agonist; an activator of the cAMP pathway; a DNA methyltransferase (DNMT) inhibitor; a nuclear receptor ligand; a GSK3 inhibitor, a MEK inhibitor, a TGFβ receptor/ALK5 inhibitor, an HDAC inhibitor; an Erk inhibitor, a ROCK inhibitor, and an FGFR inhibitor.

23.-25. (canceled)

26. The method of claim 15, comprising contacting the cell in a culture medium containing hLIF, an ALK5 inhibitor, a MEK inhibitor, and a GSK3 inhibitor.

27. A polynucleotide comprising one or more artificial pluripotency transcription factors (APTF), wherein each APTF comprises polypeptide domains encoding:
a) a nuclear localization sequence (NLS);
b) a DNA binding domain (DBD); and
c) a transcriptional activation domain (TAD),
wherein at least two of the polypeptide domains of a)-c) are heterologous polypeptide domains.

28. The polynucleotide of claim 27, wherein the APTF comprises a CPP.

29. The polynucleotide of claim 27, wherein the DBD is selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

30.-32. (canceled)

33. The polynucleotide of claim 27, wherein the TAD is selected from the group consisting of: VP16, VP64, SV40 Large T-antigen, E1A activation domain, relA, and EGFR-1.

34.-37. (canceled)

38. A polypeptide comprising one or more artificial pluripotency transcription factors (APTF), wherein each APTF comprises polypeptide domains encoding:
a) a nuclear localization sequence (NLS);
b) a DNA binding domain (DBD); and
c) a transcriptional activation domain (TAD),
wherein at least two of the polypeptide domains of a)-c) are heterologous polypeptide domains.

39. The polypeptide of claim 38, wherein the APTF comprises a CPP.

40.-43. (canceled)

44. The polypeptide of claim 38, wherein the DBD is selected from the group consisting of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, MeI-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1.

45.-47. (canceled)

48. The polypeptide of claim 38, wherein the TAD is selected from the group consisting of: VP16, VP64, SV40 Large T-antigen, E1A activation domain, relA, and EGFR-1.

49. (canceled)

50. A composition comprising a cell, one or more artificial pluripotency transcription factor polypeptides and one or more small molecule reprogramming agents.

51.-57. (canceled)
